(19) United States
(12) Patent Application Publication
(10) Pub. No.: US 2025/0092353 A1
(43) Pub. Date: Mar. 20, 2025

(54) COMMENSAL FUNGUS AND USES THEREOF

(71) Applicant: YEDA RESEARCH AND DEVELOPMENT CO. LTD., Rehovot (IL)

(72) Inventors: Steffen JUNG, Rehovot (IL); Jarmila Sekeresova KRALOVA, Rehovot (IL)

(21) Appl. No.: 18/942,735

(22) Filed: Nov. 10, 2024

Related U.S. Application Data
(63) Continuation of application No. PCT/IL2023/050470, filed on May 9, 2023.
(60) Provisional application No. 63/339,570, filed on May 9, 2022.
(60) Provisional application No. 63/450,024, filed on Mar. 5, 2023.

(51) Int. Cl. C12N 1/16; A01K 67/027; A61K 36/064; A61P 31/10; C12R 1/645

(52) U.S. Cl. CPC C12N 1/165; A01K 67/027; A61K 36/064; A61P 31/10; A01K 2207/12; A01K 2227/105; C12R 2001/645

(57) ABSTRACT

There is provided an isolated fungus belonging to the genus Kazachstania, a composition comprising same, and methods of using same, such as for preventing or treating an infection, a disease, or both.

### REFERENCE TO AN ELECTRONIC SEQUENCE LISTING

The contents of the electronic sequence listing (YEDA-P-012-US ST26 XML.xml; size: 8,982,534 bytes; and date of creation: Dec. 6, 2024) is herein incorporated by reference in its entirety.

### CROSS-REFERENCE TO RELATED APPLICATION

This application claims the benefit of priority of U.S. Provisional Patent Application No. 63/339,570, filed May 9, 2022, and of U.S. Provisional Patent Application No. 63/450,024 both titled “COMMENSAL FUNGUS AND USES THEREOF”, filed Mar. 5, 2023, all of which are hereby incorporated by reference in their entirety.

### FIELD OF INVENTION

The present invention relates to inter alia an isolated fungus, composition comprising same, and a method of using same, such as for preventing or treating a disease, an infection, or both.

### BACKGROUND

The genetic information that defines human beings includes the genomes of the human cells but also of the commensal microbiome. While the analysis of this metagenome has focused mainly on the abundant bacterial commensals, mucosal surfaces are also inhabited by fungi. However, the role of the mycobiota and their contributions to host fitness are less well understood. This is partially due to the underrepresentation of fungi in the microbiome of animal models that are kept under strict hygienic conditions.

As one of the most common human fungal pathogens, Candida albicans causes hundreds of millions of symptomatic infections each year. Pathologies are frequently associated with immuno-deficiencies and range from superficial irritations of the skin and mucosae to life-threatening invasive infections of internal organs. In addition, inborn errors of IL-17 immunity are strongly linked to chronic mucocutaneous candidiasis (CMC). Fungal dissemination, leading to systemic infection, is believed to originate from the gut, where C. albicans normally reside as a harmless commensal. Candidiasis has been linked to filamentation of the fungus, which is strictly associated with the expression of the cytolytic peptide toxin candidalysin that promotes barrier damage. Human individuals are colonized in childhood, and clonal fungal populations persist over their lifetime, mostly without any symptoms. Emerging evidence suggests that fungal colonization in fact rather benefits, than harms the host and that the gut mycobiota contributes to improving mammalian immunity. Commensal fungi, specifically C. albicans, were shown to affect the composition of the myeloid innate immune compartment and elicit cellular and humoral immunity. Insights into the diverse interactions of fungi with the mammalian hosts and other fungi, but also communication with bacterial commensals, could significantly aid the understanding of host physiology. Moreover, a better understanding might allow harnessing the impact of commensal fungi on human immunity for therapeutic purposes. The host-fungi interface remains, however, incompletely understood, not the least due to the lack of suitable experimental animal models and our limited insight into fungal commensalism.

The dominant human fungal commensal, C. albicans, has also been reported as part of the mycobiota of wild mice. However, animals kept under special pathogen-free (SPF) conditions mostly lack C. albicans and generally harbor poorly developed mycobiota that also differ considerably between vendors. Laboratory mice generally even resist C. albicans colonization unless subjected to antibiotics (Abx), which are believed to be required to neutralize inhibiting bacteria, including Lactobacillae.

There is still a great need for effective and safe treatment for C. albicans infections, such as by utilizing commensal fungi.

### SUMMARY

The present invention, in some embodiments, is based, at least in part, on the serendipitous identification of a novel fungal commensal of the Kazachstania genus that efficiently colonizes laboratory animals kept in SPF facilities without prior Abx conditioning. The isolated commensal fungus, disclosed herein, is termed K. weizmannii, and was shown to outcompete C. albicans during competitive seeding and even expelled C. albicans from stably colonized animals. Further, the current invention, in some embodiments, is based, at least in part, on the finding that unlike C. albicans, the non-filamenting K. weizmannii fungus did not disseminate or cause pathology in immune-suppressed animals. In fact, the inventors showed that K. weizmannii posed an inter-fungal competition that reduced the intestinal C. albicans load of mice mitigated fatal candidiasis.

The present invention, in some embodiments, is further based, at least in part, on the findings that colonization of fungi of the Kazachstania clade was found in some individuals to show inverse correlation with C. albicans abundance in the gut.

Therefore, the isolated fungus disclosed herein is suggested as a robust competitive commensal to C. albicans, and a method for mitigating systemic fungal pathology, is devised therewith.

According to the first aspect, there is provided an isolated fungus belonging to the genus Kazachstania and deposited at the ATCC under the deposit number PTA-127318.

According to another aspect, there is provided a composition comprising the isolated fungus, and a pharmaceutically acceptable carrier.

According to another aspect, there is provided a composition comprising a fungus, wherein at least 90% of the fungus is the isolated fungus disclosed herein.

According to another aspect, there is provided a method for preventing or treating an autoimmune disease in a subject in need thereof, the method comprising administering to the subject a therapeutically effective amount of a pharmaceutical composition comprising the isolated fungus disclose herein, thereby preventing or treating an autoimmune disease in the subject.

According to another aspect, there is provided a method for preventing or treating a fungal infection in a subject in need thereof, the method comprising administering to the subject a therapeutically effective amount of a pharmaceutical composition comprising the isolated fungus disclosed herein.

According to another aspect, there is provided non-human animal subject comprising the isolated fungus disclosed herein.

According to another aspect, there is provided a method for producing the non-human animal subject disclosed herein, comprising colonizing a gastrointestinal tract of a non-human animal subject with an effective amount of any one of: (a) the isolated fungus disclosed herein; and (b) the composition disclosed herein, thereby producing the non-human animal subject.

In some embodiments, the composition is for use in the treatment or prevention of a disease in a subject in need thereof.

In some embodiments, the disease comprises an autoimmune disease, an infectious disease or both.

In some embodiments, the autoimmune disease is a T helper 17 cells (Th17)-driven immunopathology.

In some embodiments, the autoimmune disease is selected from the group consisting of: multiple sclerosis, psoriasis, and asthma.

In some embodiments, the infectious disease is a fungal infection.

In some embodiments, the fungal infection comprises a Candida infection.

In some embodiments, the fungal infection comprises a Candida albicans infection.

In some embodiments, the autoimmune disease is: (i) induced T helper 17 cells (Th17) activity; (ii) characterized by increased Th17 activity; or (iii) both (i) and (ii).

In some embodiments, the Th17 activity is induced by a fungal infection.

In some embodiments, the subject is afflicted with a fungal infection.

In some embodiments, the treating comprises reducing the number or abundance of a fungus inducing said fungal infection in the gastrointestinal tract of said subject.

In some embodiments, the reducing is: (i) by at least 30% compared to control; (ii) for at least 4 days; or (iii) both (i) and (ii).

In some embodiments, the treating comprises reducing the activity, abundance, or both, of Th 17 cells in the subject.

In some embodiments, the administering comprises colonizing the gut of the subject with the isolated fungus.

In some embodiments, the non-human animal subject is gnotobiotic.

In some embodiments, the non-human animal subject is a mouse.

In some embodiments, the isolated fungus disclosed herein colonizes or is present in the gastrointestinal tract of the non-human animal subject.

In some embodiments, the method further comprises rearing/culturing the non-human animal subject under pathogen free conditions.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H include a scheme, graphs, fluorescent micrographs, and phylogenetic trees, showing the identification and characterization of K. weizmannii. (1A) Schematic non-limiting protocol of C. albicans colonization (adopted from Basu et al., 2000). (1B) Recoverable C. albicans in the feces of wt mice with Abx supplementation in the drinking water, 2 weeks after oral C. albicans inoculation. (1C) Recoverable fungi in feces of unexposed SPF mutant mice and C. albicans inoculated animals; stippled line, limit of detection. (1D) Recoverable C. albicans in the feces 2 weeks after oral inoculation—comparison of colonization between mutant and wt animals (1E) Flow cytometry and microscopical examination of fungal colonies recovered from mutant animals. (1F) Phylogenetic tree based on Maximum Parsimony of the 26S rDNA domains 1 and 2 (D1/D2) region of newly identified K. weizmannii in comparison to other yeast model species. (1G) Phylogenetic tree based on Maximum Parsimony of the 26S rDNA D1/D2 region of K. weizmannii in comparison to other Kazachstania species. (1H) Comparison of whole genome of newly identified K. weizmannii to whole genomes of Kazachstania species.

FIGS. 2A-2F include micrographs, graphs, a plot, and a curve showing in vitro culture characteristics of K. weizmannii. (2A) Representative images of C. albicans and K. weizmannii in liquid media in presence of filament-inducing conditions, n=3 independent experiments. (2B) A graph showing largest differences in growth between K. weizmannii and C. albicans with different carbon sources, based on a z-score > or <2. Differences were calculated using the normalized area under the curve (AUC) of K. weizmannii and C. albicans. Normalization of the AUC are based on the growth of both species with glucose. Size of data points corresponds to the normalized AUC of K. weizmannii with the corresponding carbon source. All measurements were done using the Biolog Phenotype MicroArrays in biological triplicates. X-axis represents the difference in AUC between K. weizmannii and C. albicans, while the x-axis shows the different carbon sources tested. The data points are color-coded according to the different types of carbon sources. (2C) A graph showing the largest differences in growth between K. weizmannii and C. albicans with different nitrogen sources, based on a z-score greater than or less than 2. Differences were calculated using AUC of K. weizmannii and C. albicans and normalized based on the growth of both species with glutamine. Size of data points corresponds to the normalized AUC of K. weizmannii with the corresponding carbon source. All measurements were performed using the Biolog Phenotype MicroArrays in biological triplicates. X-axis represents the difference in AUC between K. weizmannii and C. albicans, while the x-axis shows the different nitrogen sources tested. The data points are color-coded according to the different types of nitrogen sources. (2D) Plot of the largest differences in growth of K. weizmannii and C. albicans with different inhibitors to test their chemical sensitivity. Graph shows the largest differences in growth between K. weizmannii and C. albicans with different inhibitors, based on a z-score greater than or less than 2., calculated using AUC of K. weizmannii and C. albicans. AUC normalization was based on the growth of both species without any inhibitor at pH 5. The size of each data point corresponds to the AUC of K. weizmannii with the corresponding inhibitor. All measurements were done using the Biolog Phenotype MicroArrays for chemical sensitivity (PM21-PM25) in biological triplicates. (2E) Growth curves of C. albicans, S. cerevisiae and K. weizmannii under different Ph conditions in YPD medium; AUC, Area under curve, biological triplicates. (2F) Co-culture assay of C. albicans and K. weizmannii. GFP expressing C. albicans was detected by flowcytometry to discriminate between fungi, n=2 independent experiments.

FIGS. 3A-3M include fluorescent micrographs, graphs, and a scheme, showing K. weizmannii competition with C. albicans in colonized animals. (3A) A representative picture of a far-red fluorescent reporter Kazachstania strain (K. weizmannii ENO1-Mirf). (3B-C) Recoverable fecal K. weizmannii in colonized wild-type animals (without antibiotics) one and six months after single oral K. weizmannii ENO1-Mirf inoculation. (3D) Flow cytometric analysis of C. albicans SC5314 (ENO1-GFP), K. weizmannii (ENO1-Mirf), and mixed cultures used for oral inoculation (3E). (3E) Representative plating analysis of feces of animals inoculated with 107 yeasts of C. albicans SC5314 (ENO1-GFP) or K. weizmannii (ENO1-Mirf), and mixed cultures (see (3D)) 3 weeks after administration. (3F-3G) Flow cytometric analysis of feces of animals orally inoculated with 107 yeasts of C. albicans SC5314 (ENO1-GFP), K. weizmannii (ENO1-Mirf), and mixed cultures (see (D)) 3 weeks after administration. Quantification (n=3-5 mice per group, 4 independent experiments) (see (3D). (3H) Schematic non-limiting outline of a co-housing experiment. (3I) Representative flow cytometric feces analysis of colonized mice 4 weeks after initial colonization (before cohousing) and 2 weeks after co-housing. (N=4 per group). (3J) Percentages of respective labelled fungi in feces of C. albicans, K. weizmannii colonized animals, before and after co-housing in Abx presence (except for day 48 and 58); antibiotics withdrawal on day 46 of cohousing, n=4 per group, representative of 2 independent experiments. (3K) Flow cytometric analysis of feces of germfree animals 1 week after oral inoculation of single fungi cultures or mixed C. albicans SC5314 (ENO1-GFP)/K. weizmannii (ENO1-Mirf) culture. (3L) Percentages and (3M) amount of C. albicans and K. weizmannii recovered from feces of germ-free animals colonized with C. albicans SC5314 (C), K. weizmannii (K), or mixed culture (C/K) compared to Abx-treated SPF animals 1 week after administration as determined by flow cytometry analysis (3K); representative of 2 independent experiments.

FIGS. 4A-4F include graphs showing humoral and cellular immune responses to C. albicans and Kazachstania sp. (4A) Flow cytometric analysis of myeloid blood cell compartment of mice 4 weeks colonized with C. albicans or K. weizmannii. Neutrophils, classical and non-classical monocytes are defined as Ly6G+CD 115−, Ly6C+CD 115+, and Ly6C+CD 115+ cells, respectively. N=8-21 mice, pooled from 3 independent experiments. Gating strategy, left, results, right. (4B) Flow cytometric analysis of humoral anti-fungal reactivity. Gating strategy for the determination of serum immunoglobulin binding to cultured C. albicans or K. weizmannii. (4C) Anti-C. albicans and K. weizmannii IgA and IgG serum reactivity of single-colonized animals, measured as shown in (4B). Note that most but not all animals harbor cross-reactive sera. (4D) Representative gating strategy of RORγt+ T cells (left); percentage of RORγt+ cells among TCRβ CD4+ T cells 5 weeks upon C. albicans or K. weizmannii colonization, n=3-5 mice per group, 3 independent experiments (right). (4E) Representative gating strategy of RORγt+ T cells (left); percentage of RORγt+ cells among TCRβ CD4+ T cells 5 weeks upon C. albicans or K. weizmannii colonization, n=3-5 mice per group, 3 independent experiments (right). (4F) Epithelial cell (EC) coculture assay of C. albicans, S. cerevisiae, and K. weizmannii. EC damage was assessed using the LDH assay as described in (Allert et al., 2018).

FIGS. 5A-5I include a scheme, graphs, micrographs, and fluorescent micrographs, showing that commensal C. albicans but not K. weizmannii causes pathology in immunosuppressed animals. (5A) A non-limiting schematic outline of experimental set up for immunosuppression protocol. (5B) A weight curve of C. albicans-colonized mice and uncolonized controls upon cortisone 21-acetate injections, n=5 mice per group. (5C) Representative pictures of tongue candidiasis observed in C. albicans-colonized mice day 10 post immunosuppression. (5D) A representative picture of C. albicans-induced kidney pathology. (5E) A weight loss curve, comparison of C. albicans and K. weizmannii colonized mice, n=4-7 mice per group, 4 independent experiments, error bar represents SEM. (5F) Percent of initial weight in the endpoint of the experiment, graph represents mean with SD. (5G) Representative pictures of C. albicans and K. weizmannii recoverable by plating of feces (10 ng) and kidneys (10 mg) of C. albicans or K. weizmannii colonized animals (with Abx) 10 days after immunosuppression. (5H) Pathology score based on microscopic and macroscopic evaluation of kidneys, score scheme. (5I) Weight loss comparison between K. weizmannii- and non-colonized animals. n=5 per group.

FIGS. 6A-6F include a scheme, graphs, micrographs, and fluorescent micrographs showing that K. weizmannii mitigates C. albicans-induced pathology in immunosuppressed mice. (6A) Recovered C. albicans in feces (during continuous Abx treatment), and upon Abx withdrawal, as analyzed by flow cytometry and cultivation. (6B) A weigh monitoring curve following immunosuppression of C. albicans-colonized animals kept on Abx or withdrawn from Abx. (6C) Representative pictures of recoverable C. albicans and K. weizmannii from feces of C. albicans-colonized animals with or without K. weizmannii supplementation in their drinking water, analyzed by flow cytometry and cultivation. (6D) A weight monitoring curve (% initial weight), comparison of immunosuppressed animals following C. albicans-colonization and K. weizmannii-outcompeted C. albicans colonization. (6E) Representative pictures of kidneys (PAS staining) of immunosuppressed animals following C. albicans-colonization and K. weizmannii-outcompeted C. albicans colonization and results of cultivations of 1 mg kidney homogenate. (6F) A weight monitoring curve (% initial weight)—C. albicans and K. weizmannii-outcompeted animals, error bar SEM.

FIGS. 7A-7E include schemes, diagrams, and graphs showing the presence of Kazachstania in human metagenomes. (7A) Schematic of bioinformatic screening strategy of metagenomes. Briefly (from right to left), comparative genome analysis identified nucleotides regions that are unique to the genomes of either K. weizmannii or C. albicans species, together with genus-level regions of their ITS sequences. These regions were used to screen thousands of shotgun metagenomics collected and assembled by the Global Microbial Gene Catalogue v1.0 (2) (see methods). (7B) Venn diagram. Number of human gut metagenomics samples positive for unique genomic markers of either C. albicans or K. weizmannii out of 7,059 metagenomics datasets. Overlap of positive samples is in mixed colors. (7C) Number of human-vagina metagenomes positive for species-specific sequences of either C. albicans or K. weizmannii (ITS or unique regions). (7D) Antigen-reactive T cell enrichment (ARTE) analysis of anti-fungal CD4+ T cell reactivity in blood of human individuals. (n=10). (7E) ITS2 analysis of fecal samples of a cohort of 570 healthy individuals for presence of Candida and Kazachstania spp.

FIGS. 8A-8C include micrographs and a table showing the identification of novel fungal isolate. (8A) A gel analysis of PCR products amplifying ITS1 region of C. albicans and fungal isolate. (8B) Identification of novel fungal sp. based on ITS1 sequence. (8C) A representative example of PCR sentinel screening using PCR primers for specific ITS1 of K. weizmannii.

FIGS. 9A-9C include a sequence, a micrograph and graphs showing the generation of a K. weizmannii reporter strain. (9A) A sequence of modified fluorescent reporter miRFP670—further referred as ‘Mirf’. (9B) PCR validation of proper insertion of Mirf into K. weizmannii genome and thus generation of ENO1-Mirf fusion protein. (9C) Representative pictures of recovery of C. albicans ENO1-GFP and K. weizmannii ENO1-Mirf from feces of single-colonized followed by cohousing (started as Candida or Kazachstania).

FIGS. 10A-10D include graphs and fluorescent micrographs showing the impact of K. weizmannii colonization on microbiome. (10A) Diversity and abundance of fecal bacterial genus and species of K. weizmannii colonized vs. non-colonized group (controls) determined by 16S sequencing, collapsed to genus and species levels. (10B) Normalized abundance of fecal Lactobacillaceae (divided into specific Lactobacilli species) and Bifidobacteriaceae, determined by 16S sequencing. (10C-10D) In vivo competition between C. albicans and K. weizmannii upon co-administration in Germ-free animals and in antibiotics treated wild type mice: FACS and cultivation analysis of culture inoculate for p.o. administration. Representative picture of recovery of C. albicans ENO1-GFP and Kazachstania ENO1-Mirf from feces of colonized animals using flow cytometry and cultivation.

FIGS. 11A-11B include graphs and micrographs showing analysis of C. albicans-colonized immunosuppressed animals. (11A) Absolute weight monitoring (grams) of individual mice in C. albicans colonization, K. weizmannii colonized and non-colonized group. (11B) Representative pictures of kidneys and tongues (PAS staining) of immunosuppressed non-colonized and Kazachstania-colonized animals in end time point (day 10).

FIGS. 12A-12B include graphs showing analysis of K. weizmannii and C. albicans-colonized immunosuppressed animals. (12A) Absolute weight (grams) monitoring, comparison of immunosuppressed animals following C. albicans-colonization and K. weizmannii-outcompeted C. albicans colonization, monitoring till the end of experiment (day 10). (12B) Absolute weight (grams) monitoring showing the prolonged course of immunosuppressive treatment (mice sacrificed upon >20% weight loss), comparison of immunosuppressed animals following C. albicans-colonization and K. weizmannii-outcompeted C. albicans colonization, K. weizmannii colonized and non-colonized animals.

FIGS. 13A-13B include tables showing metagenome analysis. (13A) Metadata and read counts of 22 metagenomes from human vaginal origin, in which either K. weizmannii or C. albicans were identified. (13B) Metadata and read counts of 32 metagenomes from human gut origin, in which K. weizmannii was identified. Number of mapped reads of K. weizmannii or C. albicans is noted, along with metadata information collected on the metagenomics projects from NCBI Sequence Read Archive (SRA) data.

### DETAILED DESCRIPTION

### Isolated Fungus, and Composition

According to some embodiments, there is provided an isolated fungus deposited at the ATCC under the deposit number PTA-127318.

According to some embodiments, there is provided an isolated fungus belonging to the genus Kazachstania.

In some embodiments, the isolated fungus deposited at the ATCC under the deposit number PTA-127318 belongs to the genus Kazachstania.

In some embodiments, the isolated fungus is a type of yeast.

In some embodiments, the isolated fungus comprises at least one nucleic acid sequence as set forth in any one of SEQ ID Nos: 1-33.

According to some embodiments, there is provided a composition comprising a fungus. In some embodiments, the fungus comprises the isolated fungus disclosed herein. In some embodiments, at least 20%, at least 30%, at least 40%, at least 50%, at least 60%, at least 70%, at least 80%, at least 90%, at least 95%, at least 97%, at least 99%, or 100% of the fungus is the isolated fungus disclosed herein, or any value and range therebetween. Each possibility represents a separate embodiment of the invention.

In some embodiments, 20-100%, 40100%, 60-100%, 70-100%, or 80-100% of the fungus is the isolated fungus disclosed herein. Each possibility represents a separate embodiment of the invention.

In some embodiments, there is provided a fungal composition, wherein the isolated fungus disclosed herein constitutes at least 20%, at least 30%, at least 40%, at least 50%, at least 60%, at least 70%, at least 80%, at least 90%, at least 95%, at least 97%, at least 99%, or 100% of fungi of the fungal composition, or any value and range therebetween. Each possibility represents a separate embodiment of the invention.

In some embodiments, the isolated fungus disclosed herein constitutes 20-100%, 40100%, 60-100%, 70-100%, or 80-100% of fungi of the fungal composition. Each possibility represents a separate embodiment of the invention.

In some embodiments, the composition is for use in the treatment or prevention of a disease in a subject in need thereof.

In some embodiments, the composition is for use in the preparation of a medicament or a product for the treatment or prevention of a disease in a subject in need thereof.

In some embodiments, the disease comprises an autoimmune disease, an infectious disease or both.

In some embodiments, an autoimmune disease comprises a T helper 17 cells (Th17)-derived immunopathology. In some embodiments, an autoimmune disease comprises: (i) induced T helper 17 cells (Th17) activity; (ii) characterized by increased Th17 activity; or (iii) both (i) and (ii).

In some embodiments, Th17 activity is induced by a fungal infection. In some embodiments, Th17 activity comprises expression, translation, protein secretion, or any combination thereof, of at least one cytokine or a gene encoding thereof. In some embodiments, at least one cytokine comprises: interleukin (IL)-17A, IL-17F, IL-21, IL-22, CCL20, or any combination thereof.

In some embodiments, expression comprises gene expression, e.g., transcription of a gene to a messenger RNA (mRNA). In some embodiments, translation comprises production of a protein product encoded according to the gene as disclosed, or an mRNA transcript thereof.

Methods for determining any one of expression, translation, protein secretion, are common and would be apparent to one of skill in the art. Such methods include, but are not limited to, polymerase chain reaction (PCR), quantitative PCR (qPCR), western blot, enzyme linked immunosorbent assay (ELISA), flow cytometry, and others, some of which are exemplified herein below.

As used herein, the term “T helper 17 cells (Th17)-derived immunopathology” encompasses any disease, such as an autoimmune disease, that involves, is induced, comprises, characterized, enhanced, exaggerated, propagated, incited, or any combination thereof, Th17 cells.

In some embodiments, an autoimmune disease comprises multiple sclerosis, psoriasis, asthma, or any combination thereof.

In some embodiments, an infectious disease comprises or is an infection. In some embodiments, the infectious disease is a fungal infection. In some embodiments, the disease involves, is induced, comprises, characterized, enhanced, exaggerated, propagated, incited, or any combination thereof, by a fungus.

In some embodiments, a fungal infection comprises a Candida infection.

In some embodiments, a fungal infection comprises a Candida albicans infection.

In some embodiments, the composition is a synthetic or an artificial composition.

In some embodiments, the composition is formulated for colonization of a gut of a subject. In some embodiments, the composition is formulated for rectal delivery, oral delivery, parenteral delivery, or any combination thereof.

As used herein, the terms “synthetic” or “artificial” are interchangeable and refer to a manmade composition, such as in vitro grown, cultured, or formulated composition, e.g., in a lab or a comparable facility.

In some embodiments, the composition further comprises an acceptable carrier. In some embodiments, the carrier comprises or is a pharmaceutically acceptable carrier.

As used herein, the term “carrier”, “excipient”, or “adjuvant” refers to any component of a composition, that is not the active agent, such as, but not limited to the bacterial consortium disclosed herein.

In some embodiments, the carrier is a physiologically acceptable carrier. In one embodiment, the phrases “physiologically acceptable carrier” and “pharmaceutically acceptable carrier” which be interchangeably used refer to a carrier or a diluent that does not cause significant irritation to an organism and does not abrogate the biological activity and properties of the administered compound. An adjuvant is included under these phrases. In one embodiment, “excipient” refers to an inert substance added to a pharmaceutical composition to further facilitate administration of an active ingredient. In one embodiment, excipients include calcium carbonate, calcium phosphate, various sugars and types of starch, cellulose derivatives, gelatin, vegetable oils and polyethylene glycols.

As used herein, the term “carrier”, “excipient”, or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8^{th} Ed., Gilman et al. Eds. Pergamon Press (1990); Remington's Pharmaceutical Sciences, 18^{th} Ed., Mack Publishing Co., Easton, Pa. (1990); and Remington: The Science and Practice of Pharmacy, 21^{st} Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

### Methods of Use

According to some embodiments, there is provided a method for preventing or treating an autoimmune disease in a subject in need thereof.

According to some embodiments, there is provided a method for preventing or treating a fungal infection in a subject in need thereof.

In some embodiments, the method comprises administering to the subject a therapeutically effective amount of a pharmaceutical composition comprising the isolated fungus disclosed herein.

In some embodiments, the subject is afflicted with a fungal infection.

In some embodiments, the subject is a mammal subject, such as, but not limited to a human subject.

In some embodiments, treating comprises reducing the number or abundance of a fungus inducing the fungal infection in the subject.

In some embodiments, the number or abundance of a fungus inducing the fungal infection is reduced in the gastrointestinal tract (GI) of the subject.

In some embodiments, reduction of the number or abundance of a fungus inducing the fungal infection is determined in a sample obtained or derived from the subject.

In some embodiments, treating comprises reducing or inhibiting the activity of a fungus inducing the fungal infection in the subject. In some embodiments, reducing or inhibiting is of any one of: attachment, germination, penetration, host tissue colonization, growth, proliferation, metabolism, spore release, hyphae production, hyphae penetration, or any combination thereof, of a fungus inducing the fungal infection.

In some embodiments, the method further comprises a step of determining a number or abundance of a fungus inducing the fungal infection in the subject.

In some embodiments, the determining is in a sample obtained or derived from the subject. In some embodiments, the sample is obtained or derived from the GI of the subject.

In some embodiments, reducing is by at least 30%, at least 40%, at least 50%, at least 60%, at least 70%, at least 80%, at least 90%, at least 95%, at least 99%, or 100% compared to a control, or any value and range therebetween. Each possibility represents a separate embodiment of the invention.

In some embodiments, inhibiting comprises 100% reducing.

In some embodiments, reducing is for at least 4 days, at least 7 days, at least 14 days, at least 28 days, at least 2 months, at least 4 months, at least 6 months, or at least 1 year, or any value and range therebetween. Each possibility represents a separate embodiment of the invention.

In some embodiments, reducing is: (i) by at least 30%, at least 40%, at least 50%, at least 60%, at least 70%, at least 80%, at least 90%, at least 95%, at least 99%, or 100% compared to a control, or any value and range therebetween; and (ii) for at least 4 days, at least 7 days, at least 14 days, at least 28 days, at least 2 months, at least 4 months, at least 6 months, or at least 1 year, or any value and range therebetween. Each possibility represents a separate embodiment of the invention.

In some embodiments, a control comprises a subject not being treated according to there herein disclosed method. In some embodiments, a control comprises the subject prior to being treated or administered according to the herein disclosed method.

In some embodiments, treating comprises reducing the activity, abundance, or both, of Th 17 cells in the subject. In some embodiments, reducing is in the GI of the subject.

In some embodiments, administering comprises colonizing the gut of the subject with the isolated fungus.

In some embodiments, administering comprises retally administering. In some embodiments, administering comprises orally administering. In some embodiments, administering comprises rectally and orally administering.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. Further, treat, treatment, treating, as used herein, means any of the following: the reduction in severity of a hemostatic disorder; the prophylaxis of one or more symptoms associated with a hemostatic disorder, e.g., a bleeding episode; the reduction in the duration of a disease course of a hemostatic disorder; the amelioration of one or more symptoms associated with a hemostatic disorder; the reduction in duration of a bleeding episode associated with a hemostatic disorder; the provision of beneficial effects to a subject with a hemostatic disorder, without necessarily curing the hemostatic disorder.

As used herein, the terms “preventing” or “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, “treating” comprises ameliorating and/or preventing.

### Non-Human Animal and a Method for Preparing Same

According to some embodiments, there is provided a non-human animal subject. In some embodiments, the non-human animal subject comprises the isolated fungus disclosed herein.

In some embodiments, the non-human animal subject is gnotobiotic.

The term “gnotobiotic” would be apparent to one of skill in the art and encompasses any animal being characterized in a way that all microorganisms interacting with it are known and controlled.

In some embodiments, the non-human animal subject is a mammal. In some embodiments, the non-human animal subject is a rodent. In some embodiments, the non-human animal subject is mouse. In some embodiments, the non-human animal subject is a rat.

In some embodiments, the non-human animal subject comprises the isolated fungus disclosed herein, such as, in the gastrointestinal tract. In some embodiments, the gastrointestinal tract of the non-human animal subject is colonized with or comprises the isolated fungus disclosed herein. In some embodiments, the isolated fungus disclosed herein, colonizes or is present in the gastrointestinal tract of the non-human animal subject. In some embodiments, the isolated fungus disclosed herein, colonizes or is present in the gastrointestinal tract of the non-human animal subject, such that it constitutes at least 0.01%, 0.1%, 10% of fungus cells and/or fungal species in the gastrointestinal tract of the non-human animal subject, or any value and range therebetween. Each possibility represents a separate embodiment of the invention.

According to some embodiments, there is provided a method for producing the non-human animal subject disclosed herein.

In some embodiments, the method comprises colonizing a gastrointestinal tract of a non-human animal subject with an effective amount of the isolated fungus disclosed herein.

In some embodiments, the method comprises colonizing a gastrointestinal tract of a non-human animal subject with an effective amount of the composition disclosed herein.

In some embodiments, the method comprises colonizing a gastrointestinal tract of a non-human animal subject with an effective amount of the isolated fungus disclosed herein, and the composition disclosed herein.

In some embodiments, colonizing comprises rectally administering, orally administering, parenterally administering, or any combination thereof.

In some embodiments, the method further comprises rearing and/or culturing the non-human animal subject disclosed herein.

In some embodiments, the rearing and/or culturing is performed under pathogen free conditions, such as, specific pathogen free (SPF). In some embodiments, the rearing and/or culturing is performed under sterile conditions.

### Kit and Method for Determination

According to another aspect, there is provided a kit for genotypic determination of the presence of the isolated fungus disclosed herein. In some embodiments, the determination is in a sample. In some embodiments, the determination is in vitro or ex vivo determination, e.g., in a tube, a plate, or any equivalent thereof being apparent to a person of skill in the art as means for genotypic determination. In another embodiment, the present invention further provides a pair of primers capable of amplifying a nucleic acid molecule comprising any one of SEQ ID Nos: 1-33 in a polymerase chain reaction (PCR). In another embodiment, the present invention further provides a probe capable of hybridizing to a nucleic acid molecule comprising any one of SEQ ID Nos: 1-33. In another embodiment, a kit comprises primers and PCR reagents. In another embodiment, a kit comprises the probe.

In another embodiment, the present invention further provides a method for determining the presence of isolated fungus disclosed herein in a sample, comprising the steps of: (a) extracting DNA from the sample, (b) contacting the extracted DNA from step (a) with a pair of primers capable of hybridizing to a nucleic acid molecule comprising any one of SEQ ID Nos: 1-33 in a PCR, and obtaining a PCR composition; (c) subjecting the PCR composition to PCR amplification and obtaining a product; and (d) screening the product of step (c) to the presence of the nucleic acid molecule comprising any one of SEQ ID Nos: 1-33; wherein the presence of the nucleic acid molecule comprising any one of SEQ ID Nos: 1-33 within the product indicates that an isolated fungus as disclosed herein is present in the sample, thereby determining the presence of the isolated fungus as disclosed herein in the sample. In another embodiment, the absence of the nucleic acid comprising any one of SEQ ID Nos: 1-33 indicates that the sample is devoid of the isolated fungus as disclosed herein.

In some embodiments, PCR comprises denaturing double-stranded DNA in a sample (to separate the complementary strands), annealing the primers to the dissociated DNA strands, and extension reaction from the primers catalyzed by a thermostable DNA polymerase, the cycle is then repeated.

In some embodiments, a pair of DNA primers as described herein are specifically complementary to and hybridizing with opposite strands DNA with one to the left (5′) and one to the right (3′) of the target sequence within the nucleic acid sequence set forth in any one of SEQ ID Nos: 1-33, to be amplified. In some embodiments, the nucleic acid molecule set forth in any one of SEQ ID Nos: 1-33, is a specific marker of the isolated fungus disclosed herein. In some embodiments, the existence of a nucleic acid molecule comprising any one of SEQ ID Nos: 1-33 in a sample, or DNA extracted from a sample as described herein provides direct evidence for the presence of isolated fungus as described herein. In some embodiments, DNA is total DNA.

In one embodiment, a kit as described herein further comprises a DNA polymerase. In one embodiment, a kit as described herein further comprises a thermostable DNA polymerase.

As used herein, the term “screening” comprises identifying, isolating, enriching or any combination thereof. In one embodiment, methods for visualizing the nucleic acid molecule as described herein or the amplicons generated in the PCR is gel electrophoresis in polyacrylamide or agarose, followed by ethidium bromide staining. The observed sizes of the amplified target fragment—the nucleic acid molecule as described herein, should be identical to the predicted size based on the known nucleotide sequence as described and exemplified. In one embodiment, methods for visualizing the nucleic acid molecule as described herein or the amplicons generated in the PCR comprise Southern blot probing, dot-blots, or any known DNA hybridization technique wherein the nucleic acid molecule as described herein is utilized as a probe. In one embodiment, methods for visualizing the nucleic acid molecule as described herein or the amplicons generated in the PCR comprise a dissociation curve, a high-resolution melting curve, or any other DNA melting technique known in the art.

In some embodiments, the kit as described herein comprises a PCR buffer. In some embodiments, a PCR buffer comprises: 5 to 100 mM Tris-HCl and 20 to 100 mM KCl. In some embodiments, a PCR buffer further comprises 10 to 100 mM Magnesium Chloride. In some embodiments, the kit as described herein comprise a dNTP mixture. In some embodiments, the kit as described herein comprises DNA Polymerase such as but not limited to Taq DNA Polymerase. In some embodiments, the kit as described herein comprises distilled water.

An example deposit of the Kazachstania weizmannii of budding yeast has been deposited under ATCC PTA-127318.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### DEPOSIT INFORMATION

A deposit of Kazachstania weizmannii budding yeast disclosed above and recited in the claims, has been made with the ATCC® Patent Depository (10801 University Boulevard, Manassas, Virginia 20110 USA) on Jun. 22, 2022. The accession number for those deposited budding yeast is PTA-127318. Upon issuance of a patent, all restrictions upon the deposit will be removed. The deposit has been accepted under the Budapest Treaty and will be maintained in the depository for a period of 30 years, or 5 years after the last request, or for the effective life of the patent, whichever is longer, and will be replaced if necessary during that period.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Mice

This study involved wildtype and mutant mice (Cx3cr1Cre:Il23afl/fl (59, 60), all on C57BL/6 background. Unless indicated otherwise, animals were maintained in a specific-pathogen-free facility with chow and water provided ad libitum. Experiments were performed using sex- and age-matched controls. Animals were handled according to protocols approved by the Weizmann Institute Animal Care Committee as per international guidelines.

### Microbes

The recombinant C. albicans SC5314 strain expressing ENO1-GFP fusion protein is as described in Gonia et al., 2017. K. weizmannii was first cultivated from feces of mice housed in the Weizmann Institute SPF facility. The fluorescent K. weizmannii strain with a fusion of ENO1 to the modified miRFP670 (herein Kazachstania-Mirf) was generated using Crispr/Cas9 targeted mutagenesis. C. albicans strain was cultured on solid YPD media at 30° C. for 24 h-36 h. K. weizmannii and Kazachstania-Mirf were cultured on solid YPD media at 37° C.

### Fungal Gut Colonization

To establish intestinal colonization with C. albicans and K. weizmannii the drinking water of mice was supplemented with ampicillin (1 mg/mL, Ampicillin sodium salt 5 G SIGMA cat. 9518) 2-3 days prior to oral fungal inoculation. Mice were maintained on Abx-supplemented drinking water throughout the whole experiment, unless stated otherwise. For oral inoculation, C. albicans and K. weizmannii were grown on solid YPD media 30° C., or 37° C. respectively. Cultures were washed with PBS, and 107 yeast cells in 30 μl PBS were administered dropwise into the mouths of mice.

### Fungal-Mediated Pathology

Stably fungal-colonized mice were 5×s.c injected with 225 mg kg-1 with cortisone 21-acetate, following the scheme of injection every other day as described previously (Solis and Filler, 2012). Mice were monitored for weight loss. Following sacrifice, organs and feces were collected for histological examination, flow cytometry and fungal cultivations). If the weight dropped to 20% of the starting weight, mice were sacrificed according to IACUC protocol.

### Determination of CFU/g Tissue/Feces

For enumerating the number of recoverable C. albicans and K. weizmannii colony forming units, individual fecal pellets or each tissue from mice was sterilely dissected, weighed, and homogenized in sterile DDW. Serial dilutions on the organ homogenate were spread onto YPD media plates and the number of individual colonies enumerated after incubation at 37° C. for 24 hours.

Fecal DNA Extraction for 16S rRNA Gene Sequencing

DNeasy Blood & Tissue Kit (cat. 69504) kit was used according to the manufacturer's instructions for fecal DNA isolation for 16S sequencing. Prior the kit isolation, frozen fecal samples were digested with proteinase K in ALT buffer of the kit at 56° C., followed by bead-beating with sterile zirconia beads (Ø0.1 mm, BioSpec, Cat. No. 11079101).

16S rRNA Gene Sequencing and Taxonomic Assignment

Demultiplexed reads were uploaded into CLC genomics workbench (Quiagen) and analyzed using their 16S microbiome pipeline. The analysis workflow consisting of quality filtration of the sequence data, and operational taxonomic unit (OTU) clustering was performed with default parameter settings. The adaptor sequence was removed and the reads with a quality score lower than 25 or length <150 were discarded. The maximum number of acceptable ambiguous nucleotides was set to 2 and the length of the reads was fixed at 200-500 bp. Chimeric sequences and singletons were detected and discarded. The remaining unique reads were used for OTU clustering, which was performed by alignment to the SILVA database at 97% sequence similarity.

Bioinformatics Analysis of 16S rRNA Gene Sequencing Data

Visualization of OTU counts was done using the Marker Data profiling pipeline of MicrobiomeAnalyst. Counts were filtered to include OTUs with minimum 2 counts (mean abundance value) and scaled to library total sum. Abundance profiles were generated after merging small taxa with counts <10 based on their median counts. In order to explore uncultured species (D6 level), the annotations of ‘uncultured bacteria’ were concatenated to their family names (D4 level). Results were used for Alpha diversity analysis using Shannon diversity, T-test on filtered data, and Beta Diversity (PCoA using Bray-Curtis index) plots.

### Bioinformatics Screening of Human Shotgun Metagenomics Datasets to Identify Fungi Species

Identification of Genomic Regions Unique to K. weizmannii and C. albicans

In order to identify K. weizmannii and C. albicans in human metagenomics datasets, the inventors first selected a set of nucleotide regions that are unique to the genome sequence of either K. weizmannii or C. albicans and were used to specifically identify these fungi in the background of other fungi, bacteria, and other species in the metagenomes. The genome assemblies of K. weizmannii and C. albicans were compared to 22 genomes (genus Kazachstania) and 11 genomes (genus Candida), respectively, using the GView Server, with analysis type ‘Unique genome’ with default parameters except for the Genetic code, where ‘Standard’ was used. Nucleotide regions unique to each target genome were collected, and further compared to the NCBI nt database using BLAST (E-value <0.0001) in order to exclude regions that are also present in bacteria or other non-fungal organisms. This search resulted in 179 nucleotide sequences specific to K. weizmannii, and 904 nucleotide sequences specific to C. albicans.

Screening Shotgun Metagenomics Datasets Using Unique Kazachstania sp. and C. albicans Queries

A dataset of 13,174 metagenomes, collected from the Global Microbial Gene Catalog v1.0 (GMGC (2)) was screened to identify sequences that originate from either Kazachstania sp. or C. albicans genomes. Each GMGC metagenome was originally stratified into habitats, and its raw nucleotide sequences were assembled into thousands of contigs (provided to us by Luis Pedro Coelho). All assembled contigs from each metagenomics sample were used as a query and were searched against the set of unique K. weizmannii or C. albicans sequences constructed as described above, as a reference. Alignments were generated using bowtie2 algorithm (version 2.3.5.1, using local mode). In order to broaden the search to genus level, all metagenomics assemblies were also mapped to a set of genus-specific rDNA and ITS sequences, extracted from K. weizmannii or C. albicans genomes (namely 25Sa, 18Sa, 5.8Sa, ITS1a, ITS2a, ETS1a, ETS2a). To avoid biases in the specificity of ITS searches, the inventors counted only sequences which mapped to ITS regions without mutations.

Genomic DNA Isolation from Fungal Cultures

Fungal pellet was dissolved in 2 ml lysis buffer (100 mM Tris pH 8.0, 50 mM EDTA, 1% SDS) and sonicated for 10 s. Three hundred (300) μl of the supernatant was transferred to 300 μl 7 M ammonium acetate pH 7.0, vortexed and incubated 5 min at 65° C. followed by 3 minutes on ice. Five hundred (500) μl chloroform was added and the solution was mixed by inverting the tube. Samples were spun 10 min at 13,000 rpm at 4° C. Three hundred (300) μl of the upper phase was transferred to 400 μl isopropanol-filled tubes and incubated for 5 min on ice. Samples were spun 10 min at 13,000 rpm at 4° C. and pellet was washed with 800 μl 70% EtOH, spun 1 min at maximum speed, air dried and dissolved in DDW.

### Genome Sequencing, Annotation, and Comparison

Sequencing and hybrid (Nanopore and Illumina) assembly were performed by SeqCenter (Pittsburgh, PA), as follows: Illumina—Sample libraries were prepared using an Illumina DNA Prep kit and IDT 10 bp UDI indices and sequenced on an Illumina NextSeq 2000 producing 2×151 bp reads. The data was demultiplexed and adapters removed using bcl2fastq [2](v2.20.0.445) (support.illumina.com/sequencing/sequencing_software/bcl2fastq-conversion-software.html). Nanopore—Samples were prepared for sequencing using Oxford Nanopore's “Genomic DNA by Ligation” kit (SQK-LSK109) and protocol. All samples were run on Nanopore R9 flow cells (R9.4.1) on a MinION. Basecalling was performed with Guppy (version 4.2.2), in high-accuracy mode (Default parameters +effbaf8). Quality control and adapter trimming was performed with porechop (https://github.com/rrwick/Porechop) version 0.2.2_seqan2.1.1 with the default parameters. Long read assembly with ONT reads was performed with flye (version 2.8). The long read assembly was polished with pilon (1.23). Annotation was performed with the Yeast Gene Annotation Pipeline (YGAP) with the Post-WGD settings, and Companion for Fungi with a reference organism of Candida glabrata CBS138. The output of both programs was compared with CD-HIT (version 4.8.1) with c=1, to reduce redundancy, and the remaining genes combined with YGAP as the base annotation, using in-house scripts. Whole genome comparison of 25 species of Kazachstania on the basis of K. weizmannii was performed with CCT (CGView Comparison Tool) (63), with a BlastN e-value of e⁻¹⁰.

### Phylogenetic Analysis

The d1d2 region of 26SrDNA of various species were aligned with both ClustalW2.1 and Muscle 3.8.31. Phylogenetic trees were constructed with Maximum likelihood and DNA parsimony, using PhyML 3.0, DNAML, DNAPARS and DNAPENNY in the Phylip package (3.697) (Felsenstein 2005). The Phylip trees were then processed through Consense. Trees with similar topologies were obtained, and the Muscle/DNAPENNY tree is shown. The tree was visualized with iTol version 6.

qPCR Quantification

Twenty to twenty five (20-25) mg of feces or tissue were surgically resected including its content, homogenized using Lysing Matrix C (MP biomedicals) with the Omni Bead Ruptor 24 (Omni international, inc) followed by DNA extraction using Quick-DNA plus kit (Zymo Research) according to manufacturer's instructions. 10 ng of isolated DNA were used for quantitative PCR reaction using Fast SYBR green Master Mix (Thermo Fisher Scientific, cat 4385614). Reaction was performed on the Quantstudio 7 Flex Real-Time PCR system (Thermo Fisher Scientific) using the fast SYBR 10 μl program. Fungal DNA content in the samples was calculated using standard curves and normalized to tissue weight. For detection of C. albicans the following primers were used: forward: 5′-GGTGTTGAGCAATACGAC-3′ (SEQ ID NO: 29), reverse: 5′-AGACCTAAGCCATTGTC-3′ (SEQ ID NO: 30). For the detection of K. weizmannii the following primers were used: forward: 5′-ATGCACGTTTTTCTGGGTGC-3′ (SEQ ID NO: 31) reverse: 5′-GTATCGCATTTCGCTGCGTT-3′ (SEQ ID NO: 32).

### In Vitro Serum Antibody Binding Assay

Blood was collected from mice at rested at RT for 1 hour, followed by centrifugation 2,000 g 10 min. Serum supernatant was collected and froze at −80° C. until use. Cultured fungi were normalized to OD600=1 in PBS supplemented with 1% bovine serum albumin and 0.01% sodium azide (PBA solution). Cultured fungi were incubated with 20× diluted mouse serum on ice for 45 minutes, then washed twice with PBA, followed by staining with anti-mouse IgA and IgG. Samples were recorded on Cytek Aurora and analyzed in FlowJo (Treestar). Antibody binding intensity was normalized to the negative controls. Isotype control, non-stained and no-serum were used as negative controls.

### Tissue Isolation for Flow Cytometry

Mesenteric and peripheral lymph nodes were aseptically resected out into sterile, ice-cold PBS and mashed manually using a 1 mL syringe plunger through a 40 μm nylon cell strainer. One hundred to two hundred (100-200) μL of mouse blood was collected from submandibular vein, resuspended in 15 μl of heparin (Sigma) to prevent coagulation, followed by lysis with 1 ml ACK buffer (8.29 g/ml NH₄Cl, and 1 g/l KHCO₃, 37.2 mg/l Na-EDTA) for 5 min RT and subsequent centrifugation.

### Cell Staining, Stimulation and Flow Cytometry and Microscopy

Methods adhered to published guidelines (Cossarizza et al., 2019). Flow cytometry samples were recorded on BD LSR Fortessa 4 lasers or Cytek Aurora followed by data analysis using the FlowJo software (Treestar).

### Immunohistochemistry and Histology

Mice were euthanized and intestines, kidneys and tongue were excised and fixed overnight in 4% paraformaldehyde at 4° C. Paraffin embedding and sectioning was performed by the institutional histology unit. For immunohistochemistry of gut samples, slides were deparaffinized by 2 washes of Xylene followed by 100%, 96%, 70% EtOH and three washes with PBS. Slides were incubated for 30 minutes in 1% hydrogen peroxide in PBS at room temperature in the dark. After washing three times in PBS, slides were boiled for 10 minutes in Citric acid buffer pH 6.0 (10 mM citric acid and 0.05% Tween-20 in DDW), then washed with PBS three times. Next, slides were blocked in BP buffer (0.3% Triton X-100, 2% horse serum and 1% bovine serum albumin in PBS) for 1 h, then incubated overnight at 4° C. with a 1:200 dilution of the primary antibody (anti-Candida) in BP buffer. In the following day, slides were washed three times in PBS and conjugated with a secondary antibody diluted 1:200 (Cy3 anti-Rabbit) in BP buffer for 1 h at 25° C. Samples were washed three times with PBS and incubated for 5 min with DAPI (1:10,000), then mounted with Immu-Mount (Epredia). Sections were imaged using Zeiss 880 confocal laser scanning microscope. Images were processed using the Zeiss Zen blue software. For histopathology of typical tongue or kidney fungal lesions in the mouse model of immunosuppression, paraffin sections were stained with periodic acid-Schiff (PAS) or H&E and slides were captured using a Panoramic SCAN II (3DHISTECH) and analyzed using CaseViewer software (3DHISTECH).

| REAGENT or RESOURCE | SOURCE | IDENTIFIER |
|---|---|---|
| IgA, PE | Invitrogen | Cat#12-4204-81 |
| IgG, Alexa Fluor ® 594 | BioLegend | Cat#405326 |
| CD45, Violet 450 | Biogems | Cat#07512-40-108 |
| CD4, Alexa Fluor ® 647 | BioLegend | Cat#100412 |
| TCR β chain, FITC | BioLegend | Cat#109206 |
| IgG1 κ Isotype Ctrl, PE | BioLegend | Cat#400407 |
| ROR gamma (t) | Invitrogen | Cat#12-69981-80 |
| TCR β chain, PE/Cyanine5 | BioLegend | Cat#109209 |
| CD274, PE/Dazzle ™ 594 | BioLegend | Cat#124323 |
| CD43, Alexa Fluor ® 700 | BioLegend | Cat#143214 |
| Ly6C, Pacific Blue ™ | BioLegend | Cat#128014 |
| Ly6G, FITC | BioLegend | Cat#127605 |
| CD115, APC/Cyanine7 | BioLegend | Cat#135532 |
| CD11b, PE | BioLegend | Cat#101207 |
| CD220, APC | BioLegend | Cat#103212 |
| Zombie Aqua ™ Fixable Viability Kit | BioLegend | Cat#423102 |
| Anti-Candida albicans | Abcam | Cat# ab53891 |
| Cy3-AffiniPure Donkey Anti-Rabbit | Jackson Immunoresearch | Cat#711165152 |
| purified CD16/32 | BioLegend | Cat#101302 |
| Calcofluor white | | |
| Cell Activation Cocktail | BioLegend | Cat#423302 |
| Brefeldin A Solution | BioLegend | Cat#420601 |
| Fixation/Permeablization Kit | BD Bioscience | Cat# 554714 |
| True-Nuclear ™ Transcription Factor Buffer Set | BioLegend | Cat#424401 |

### Antigen-Reactive T Cell Enrichment (ARTE)

PBMCs were freshly isolated from EDTA blood samples on the day of blood donation by density gradient centrifugation (Biocoll; Biochrom, Berlin, Germany). Antigen-reactive T cell enrichment (ARTE) was performed as follows. Briefly, 2×10e⁷ PBMCs were plated in RPMI-1640 medium (GIBCO), supplemented with 5% (v/v) human AB-serum (Sigma Aldrich, Schnelldorf, Germany) at a cell density of 1×10e⁷ PBMCs/2 cm² in cell culture plates and stimulated with 40 μg/ml fungal lysates for 7 hr in presence of 1 μg/ml CD40 and 1 μg/ml CD28 pure antibody (both Miltenyi Biotec, Bergisch Gladbach, Germany). 1 μg/ml Brefeldin A (Sigma Aldrich) was added for the last 2 hr. Cells were labeled with CD154-Biotin followed by anti-Biotin MicroBeads (CD154 MicroBead Kit; Miltenyi Biotec) and magnetically enriched by two sequential MS columns (Miltenyi Biotec). Surface staining was performed on the first column, followed by fixation, permeabilization (Inside stain Kit, Miltenyi Biotec) and intracellular staining on the second column. The following antibodies were used: CD4-APC-Vio770 (M-T466), CD8-VioGreen (REA734), CD14-VioGreen (REA599), CD20-VioGreen (LT20), Integrin-b7-PE-Vio770 (REA441) (all Miltenyi Biotec); CD45RA-PE-Cy5 (HI100), IFN-γ-BV785 (clone: 4S.B3) (both Biolegend); IL-17A-BV650 (clone: N49-653), IL-22-PerCP-eFluor710 (clone: IL22JOP) (both BD Biosciences). Viobility 405/520 Fixable Dye (Miltenyi Biotec) was used to exclude dead cells. Data were acquired on a LSR Fortessa (BD Bioscience, San Jose, CA, USA).

Frequencies of antigen-specific T cells were determined based on the total cell count of CD154+ T cells after enrichment, normalized to the total number of CD4+ T cells applied on the column. For each stimulation, background cells enriched from the non-stimulated control were subtracted.

### ITS2 Amplification and Sequencing of Human Stool Samples

ITS2 sequencing was used for fungal identification. Briefly, ITS2 sequencing applied to 570 human stool samples and 6 controls. PCR was performed on 10 ng of DNA per sample (or the maximum available). Three PCR batches were required with 2 wells left empty as library controls in each batch. Forward primer ITS86F 5′-759 GTGAATCATCGAATCTTTGAA-3′ (SEQ ID NO: 33) and reverse primer ITS4 with rd2 Illumina adaptor 5′-AGACGTGTGCTCTTCCGATCTTCCTCCGCTTATTGATATGC-3′ (SEQ ID NO: 94) were used for the first PCR amplification. PCR mix per sample contained 5 μl sample DNA, 0.2 M per primer (primers purchased from Sigma), 0.02 unit/μl of Phusion Hot Start II DNA Polymerase (Thermo Scientific 763 F549), 10 μl of ×5 Phusion HS HF buffer, 0.2 mM dNTPs (Larova GmbH), 31.5 μl ultra-pure water, for a total reaction volume of 50 μl. PCR conditions used were 98° C. 2 min, (98° C. 10 sec, 55° C. 15 sec, 72° C. 35 sec)×30, 72° C. 5 min. A second PCR was performed to attach Illumina adaptors and barcode per sample for 6 additional cycles. Samples from the 1^{st} PCR were diluted 10-fold and added to the PCR mix as described above. Primers of second PCR included: forward primer P5-rd1-768 ITS86F 5′-AATGATACGGCGACCACCGAGATCTACACTCTTTCCCTACACGACGCTCTTCCG ATCTGTGAATCATCGAATCTTTGAA-3′ (SEQ ID NO: 35), and reverse primer 5′-CAAGCAGAAGACGGCATACGAGATNNNNNNNNTGACTGGAGTTCAGACGTGTG CTCTTCCGATCT-3′ (SEQ ID NO: 36). Every 96 samples were combined for a single mix by adding 14 μl from each. Before mixing, an aliquot from each of the samples was run on an agarose gel. In cases where the amplified bands were very strong, samples were diluted between 5 and 20-fold before they were added to the mix. Each sample mix was cleaned with QIAquick PCR purification kit (QIAGEN, catalog #28104). Two cleaned sample mixes were then combined into a single mix of 192 samples, and size selection was performed with Agencourt AMPure XP beads (Beckman Coulter #A63881) to remove any excess primers. The beads to sample ratio was 0.85 to 1. Samples were then run in three libraries on the Miseq v3 600 cycles paired-end with 30% PhiX.

### ITS2 Sequencing Analysis

The ITS2 classification pipeline was built with Python 3.6. For each sequencing library, paired-end reads were joined using PEAR (version 0.9.10) followed by filtering of merged reads by minimum length of 80 bp and trimming of primers from both ends with cutadapt (version 1.17). Within the QIIME 2 environment (version 2018.8), Dada2 was used to create amplicon sequence variants (ASVs), then ITSx (version 1.1b1) was used to delineate ASVs to ITS2 regions (removing preceding 5.8S and trailing 28S sequences). A taxonomic naive bayesian classifier in QIIME 2 (74) was trained on the UNITE database (version 8, dynamic, sh_taxonomy_qiime_ver8_dynamic_04.02.2020.txt) and used to classify the 180 processed ASVs. 91 percent of raw reads were classified to species level (Table 1).

| | Number | Percent |
|---|---|---|
| Kingdom | 29339412 | 100 |
| Phylum | 29339412 | 100 |
| Class | 29339412 | 100 |
| Order | 29339412 | 100 |
| Family | 28453602 | 96.98082 |
| Genus | 28423083 | 96.8768 |
| Species | 26935819 | 91.80763 |

Most of the downstream analysis and plots were performed with R version 4.1.1 and phyloseq 1.34.0. ASVs were filtered by the ITSx and UNITE classifications to include fungal reads only. 119 ASVs that were classified by ITSx as fungi were included in the downstream analysis, representing over 97.5% of reads. Out of the remaining 61 ASVs that were classified by ITSx as non-fungal (Tracheophyta (T), land plants), one (fid65) was included in the downstream analysis since its classification as fungi reached all the way to species level by UNITE and was validated by NCBI BLAST to be fungal. Next, the inventors introduced two types of data normalization: (1) Library normalization, where samples were normalized to account for the difference in the average number of reads/sample per library. (2) Dilution normalization: ASV reads were multiplied by the dilution factor per sample to reflect their true original load. Next, ASVs were aggregated based on UNITE classification, to species level when possible. ASVs that could not be classified to species level, were grouped together by the lowest known phylogenetic level, and labeled “Other”. A total of 55 species were detected, 13 of which were also detected in control samples, but with a maximum of 100 reads per sample demonstrating a very mild read leakage of the higher abundant species in the library, e.g., Saccharomyces cerevisiae. Therefore, flooring of species with less than 100 reads per sample was applied. Lastly, data were aggregated by summing all reads in each taxonomic level by the associated taxa in the level above it.

### Biolog

Quantification of cytotoxicity (LDH assay). Differentiated C2BBe1 cells in 96-well plates were infected for a defined time period with 10⁴ C. albicans, S. cerevisiae and K. weizmannii. cells/well. After coincubation, epithelial damage was quantified by measuring LDH release using a cytotoxicity detection kit (Roche) according to the manufacturer's instructions.

### Filamentation Assay

C. albicans and K. weizmannii were tested for their ability to the filament in liquid filamentation conditions. Cells grown overnight in liquid YPD were centrifuged and washed 2× with PBS prior to the experiment and strains were diluted on the same OD. Yeast cells were incubated for 3 hr or 5 h in 30° C., 37° C. or 42° C. with shaking, then directly fixed with 4% PFA in PBS for 30 min, followed by wash with PBS and images were captured by bright Zeiss field microscope.

### Statistical Analysis

In all experiments, data are presented as the mean±SEM unless stated otherwise. Statistical significance was defined as P<0.05. The number of animals is indicated as n. Animals of the same age, sex, and genetic background were randomly assigned to treatment groups.

### Data Availability

The data have been deposited to BioProject accession number PRJNA949686 in the NCBI BioProject database (ncbi.nlm.nih.gov/bioproject/).

### Example 1

### Identification of a Novel Commensal Fungus in Laboratory Mice

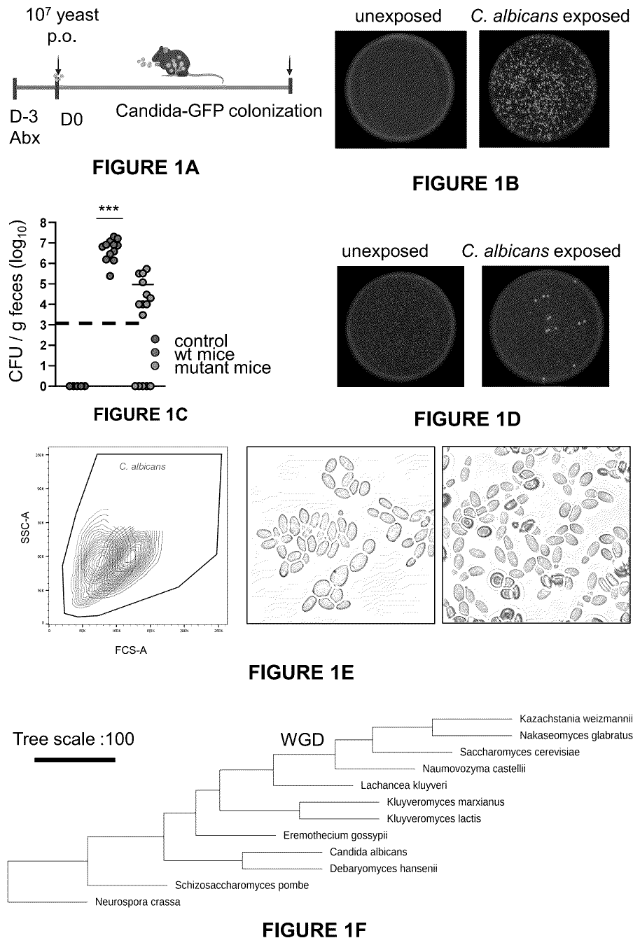

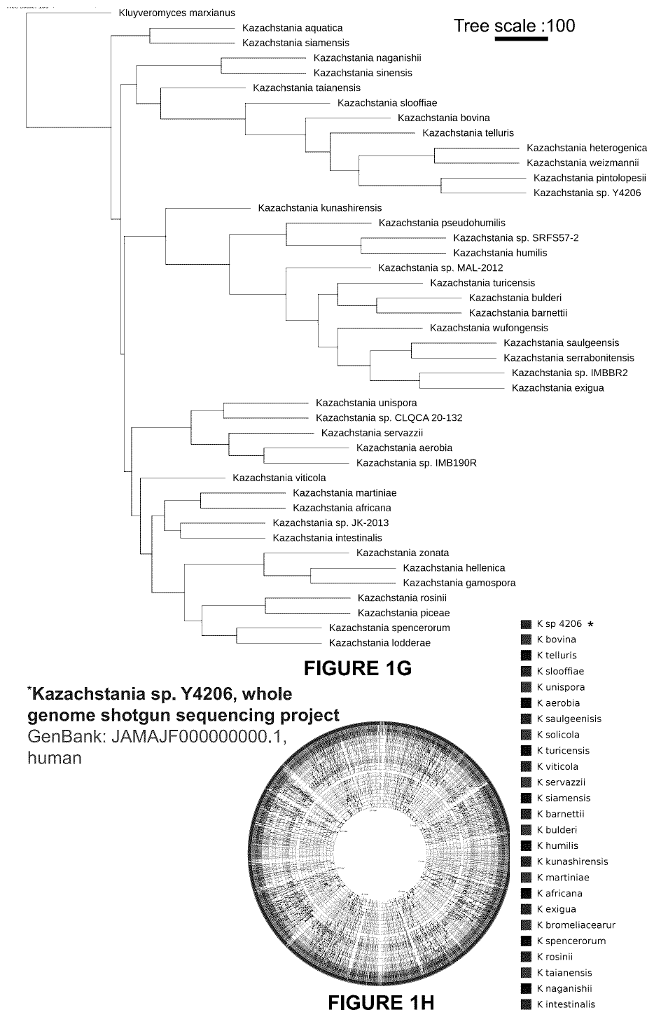

To probe for a role of myeloid cells in anti-fungal immunity, the inventors used mice that harbor a corresponding IL-23 deficiency and attempted to colonize them with C. albicans. Specifically, the inventors used a protocol involving prior conditioning of animals with antibiotics (Abx) (27) (FIG. 1A). Ampicillin-exposed wildtype (WT) mice could be readily and persistently seeded with C. albicans SC5314 harboring a GFP reporter (FIG. 1B). Surprisingly, however, the inventors consistently failed to colonize the mutant mice with C. albicans (FIG. 1C). Rather, following plating of the fecal microbiota of these animals, the inventors noted the growth of another yeast-like fungus of distinct morphology (FIGS. 1D-1E). Sequencing of DNA isolated from the plated fungus using the internal transcribed spacer (ITS) as yeast barcode tentatively identified the fungus as a member of the Kazachstania clade (FIGS. 1A-1B). This genus is composed of over 50 species found in both anthropic and non-anthropic environments and associated with sourdough production. Whole genome sequencing of the new fungus revealed the characteristic gene duplications of the Saccharomycetaceae family and established it as a novel Kazachstania strain, which the inventors term Kazachstania weizmannii. Phylogenetic analysis of 26S rDNA confirmed the assignment of K. weizmannii to the Saccharomycetaceae clade (FIG. 1F), and comparison to other Kazachstania species (FIG. 1G) placed it together with K. heterogenica, a fungus found in rodent feces, and in a sister clade with K sp. Y4206, a species isolated from human feces, and K. pintolopesii, from guinea pig peritoneal fluid. Comparison of the K. weizmannii genome with other full length Kazachstania genomes (FIG. 111) showed that K. sp Y4206 is the closest species, followed by K. bovina, K. telluris, and K. slooffiae.

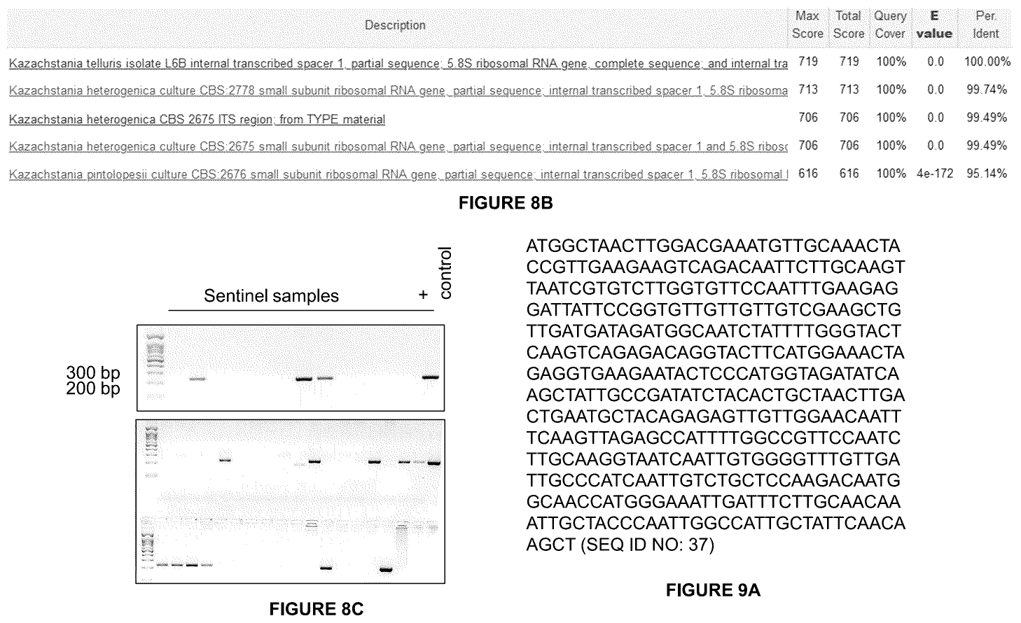

Of note, although the inventors originally discovered the novel Kazachstania species in mutant mice that might have impaired anti-fungal immunity, sentinel screening in the animal facilities with the ITS assay revealed that K. weizmannii was widespread, irrespective of animal genotypes (FIG. 8C).

### Example 2

### In Vitro Characterization of K. weizmannii

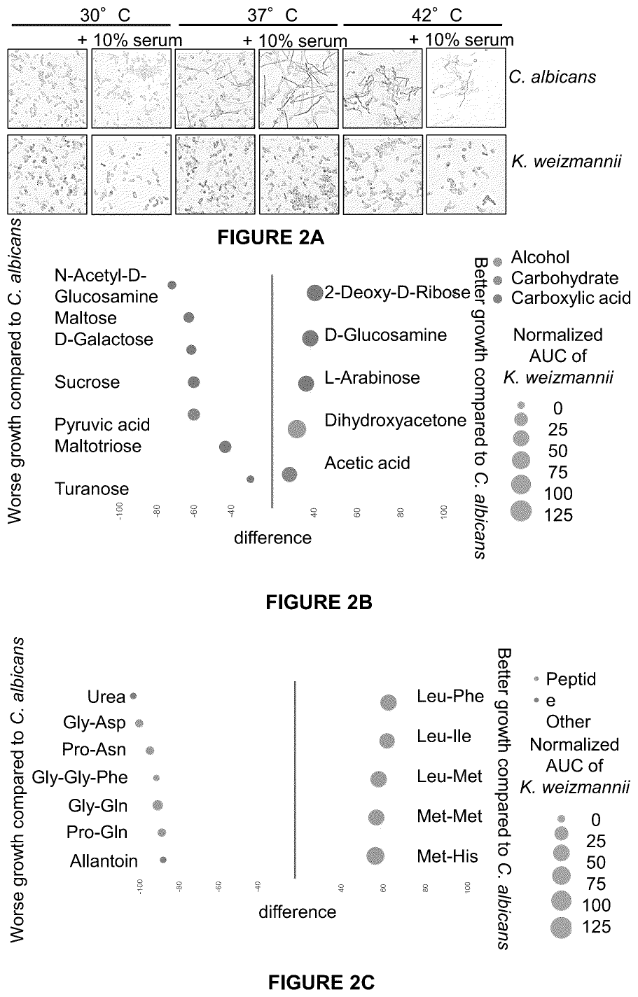

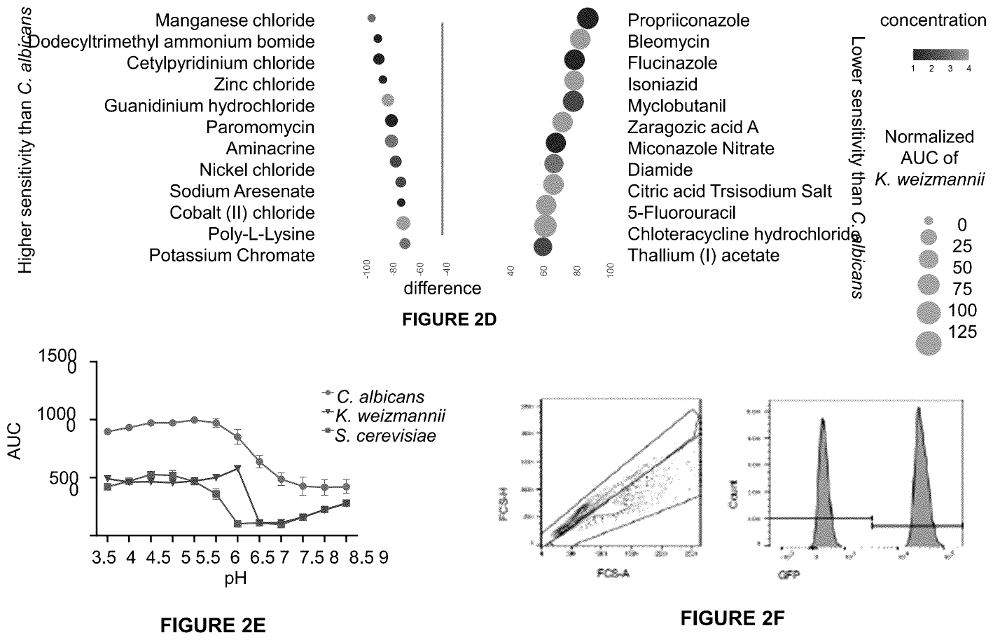

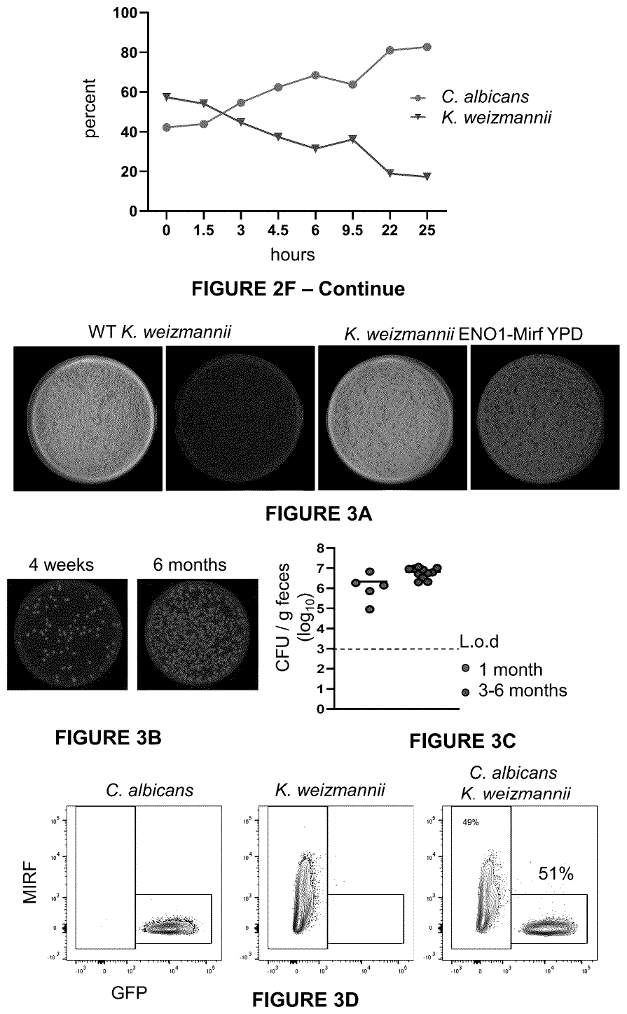

When cultured in a number of defined conditions frequently found in the human body, such as serum exposure or, neutral pH and or 37° C., C. albicans forms hyphae the same challenges did not invoke the same morphology switch in K. weizmannii (FIG. 2A). Instead, the fungus continued to grow in a yeast-like morphology.

Comprehensive Biolog analysis revealed preferences for carbon and nitrogen sources of C. albicans and K. weizmannii (FIGS. 2B-2C). K. weizmannii was for instance superior in growing with 2-Deoxy-D-Ribose and D-Glucosamine, as well as leucine dipeptides, while C. albicans could better thrive on maltose, D-galactose, urea and glycine dipeptides. The two fungi also displayed differential resistance to chemical inhibitors (FIG. 2D). Specifically, K. weizmannii showed as compared to C. albicans relative resistance to propriconazole and fucinazole, while the yeast was more sensitive to chlorides and bromides. When cultured in YPD media C. albicans and K. weizmannii displayed similar pH optima (FIG. 2E). Interestingly and in contrast to the observation for the gut, in in vitro cocultures, C. albicans and K. weizmannii grew together without interference (FIG. 2F).

### Example 3

### K. weizmannii is a Murine Commensal that Antagonizes C. albicans Colonization

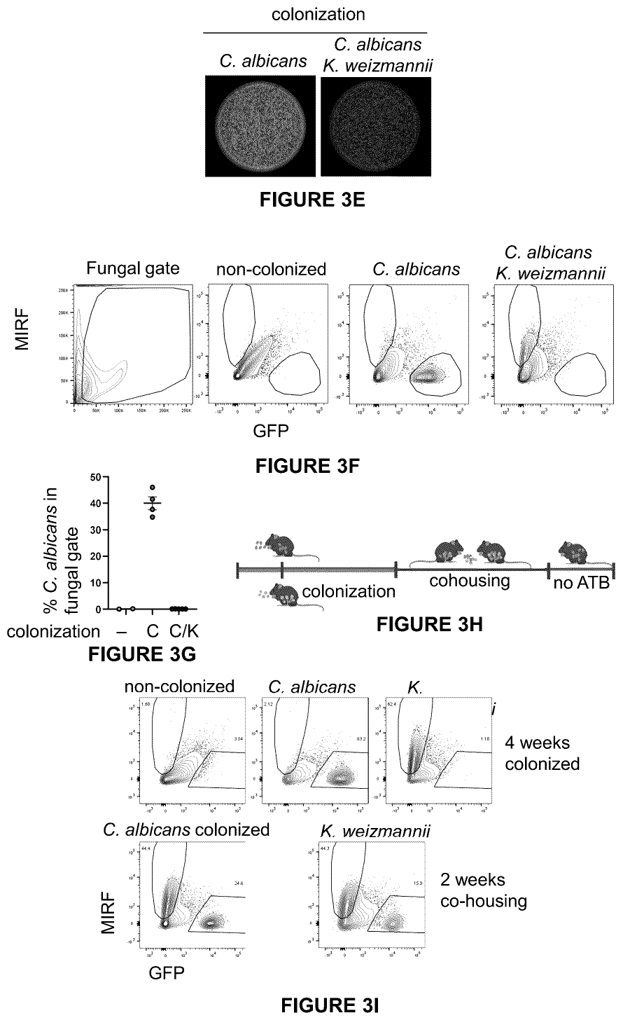

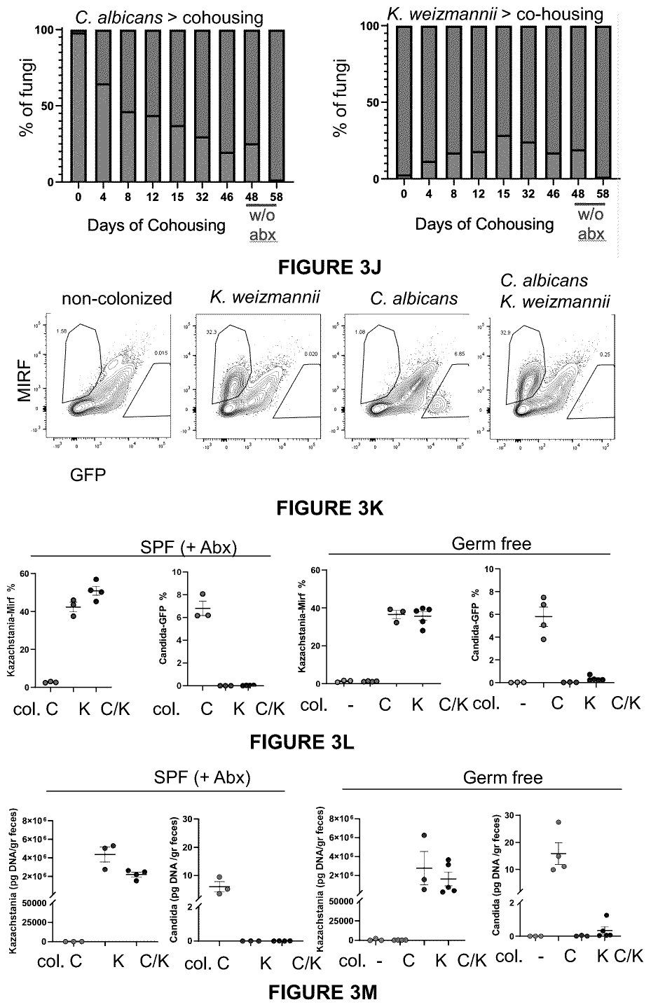

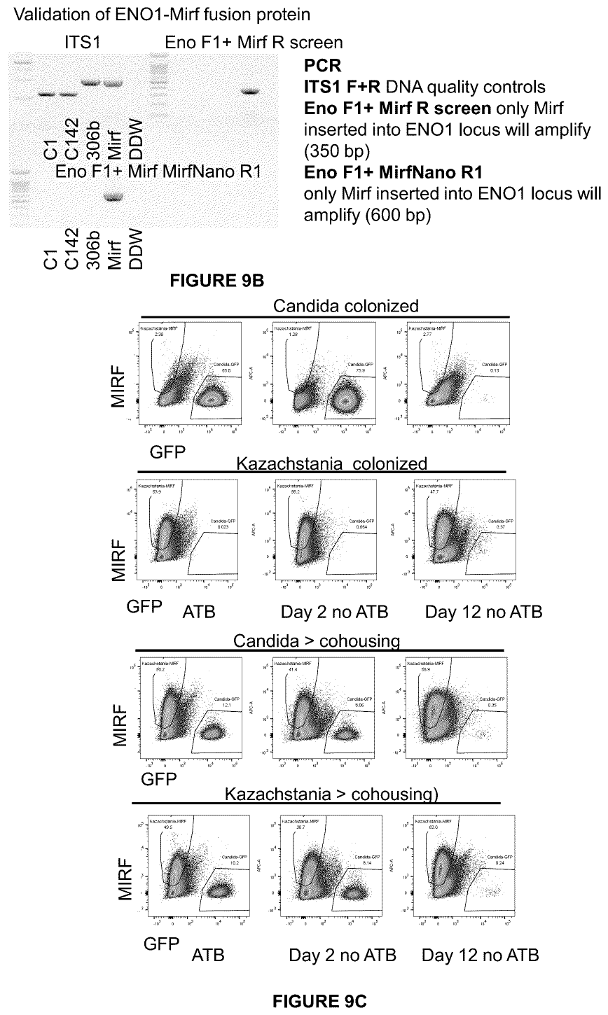

To facilitate the comparative analysis of K. weizmannii and C. albicans SC5314, the inventors generated a K. weizmannii strain harboring a gene encoding a red fluorescent reporter in the enolase 1 (ENO-1) locus, mimicking the C. albicans SC5314-GFP configuration (FIGS. 3A, 3C, and 9B-9C).

Colonization of WT animals by K. weizmannii did not require prior Abx treatment, in contrast to the colonization with C. albicans (FIGS. 3B-3C). C57Bl/6 mice could be readily colonized by oral K. weizmannii inoculation or cohousing with animals bearing the fungus. Interestingly, and in line with the original observation disclosed herein, when Abx-treated mice were co-inoculated with a 1:1 mixture of K. weizmannii and C. albicans, Candida colonization was prevented (FIGS. 3D-3G). To test if K. weizmannii would also outcompete established commensal C. albicans, the inventors stably colonized mice with C. albicans (on Abx) and then cohoused the animals with mice harboring K. weizmannii (FIG. 3H). Coprophagy led to the progressive rapid ousting of C. albicans by K. weizmannii, leaving only a residual C. albicans population. Colonization with C. albicans was further reduced upon Abx withdrawal (FIGS. 3I-3J).

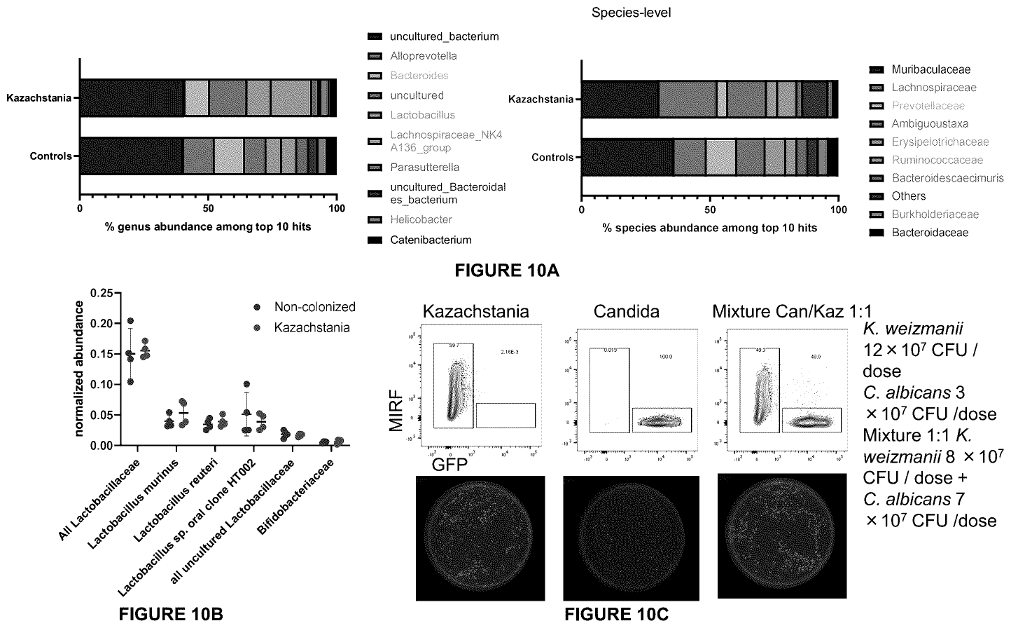

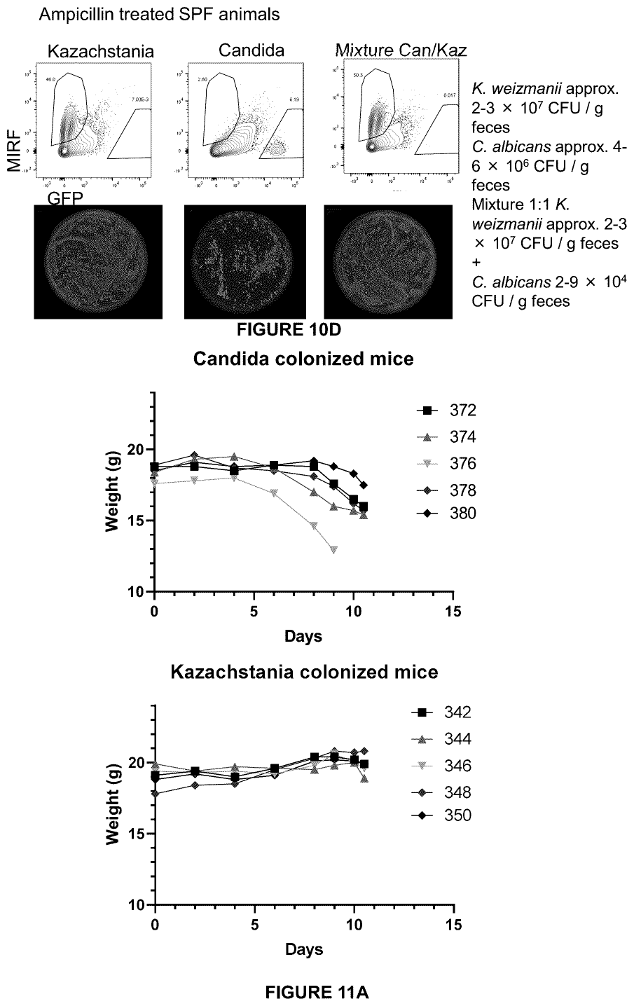

C. albicans colonization of mice is sensitive to the microbiome composition. The competition between the two fungi that the inventors observed could hence be due to alterations of the bacterial landscape. Comparison of the microbiome of K. weizmannii—colonized mice and non-colonized controls using 16S sequencing revealed, however, only minor consistent changes (FIG. 10A). Moreover, also the abundance of Lactobacillae that are known to compete with C. albicans and to impede colonization of the murine gut, was unaltered (FIG. 10B). To further probe the potential involvement of bacterial microbiota in fungal competition, the inventors orally inoculated germ-free animals with a mixture of the two fungi. Also in these mice, K. weizmannii prevented efficient C. albicans colonization (FIGS. 3K-3M, and 10C-10D). Collectively, these data establish that K. weizmannii can outcompete C. albicans including previously colonized animals and that the observed inter-fungal competition is independent of bacterial components.

### Example 4

### Comparative Analysis of the Host Immune Response to C. albicans and K. weizmannii

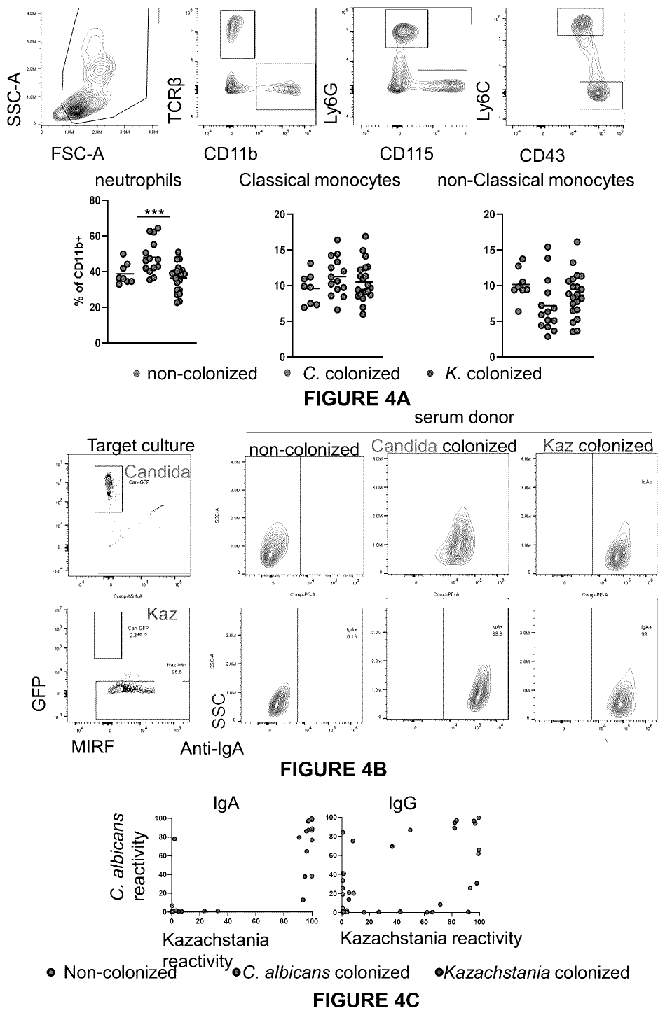

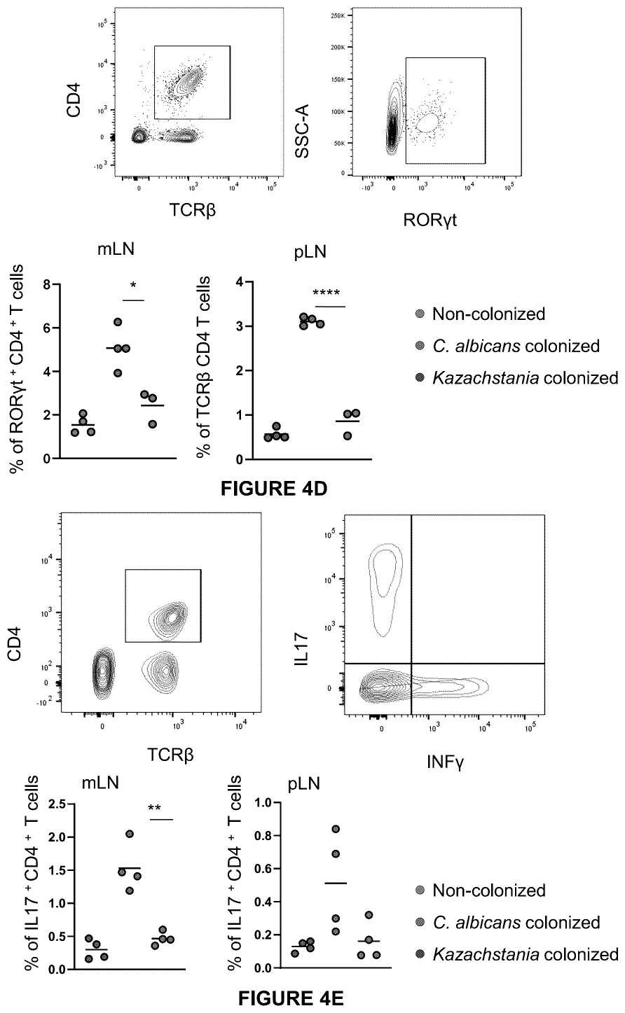

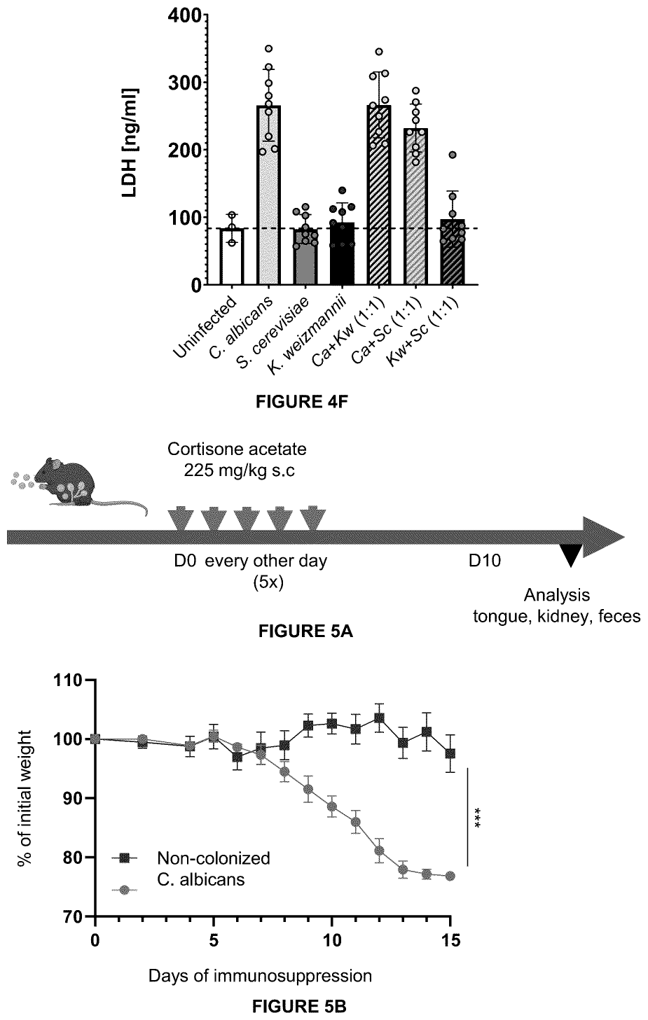

Fungi are known to affect host granulopoiesis and induce both humoral and cellular immunity. In line with these reports, Abx-treated animals colonized with C. albicans showed an expanded neutrophil compartment in the blood. In contrast, K. weizmannii-colonized animals displayed no significantly altered abundance of granulocytes, classical or non-classical monocytes (FIG. 4A).

Intestinal IgA responses to C. albicans were proposed to balance commensalism vs. pathogenicity by controlling the critical morphological hyphae-to-yeast switch of the fungus. To assess humoral immunity against the two fungal commensals, the inventors analyzed sera of colonized animals for reactivity to cultured K. weizmannii or C. albicans. Colonization with either yeast induced robust anti-fungal serum IgA and IgG titers in most animals. (FIGS. 4B-4C). The induced antibodies were mostly, but not always, cross-reactive between the two fungal species.

Mucosa-associated fungi, and specifically C. albicans have been shown to induce Th17 type cellular immune response. Accordingly, C. albicans-colonized animals displayed an expansion of Th17 cells in gut mucosa-associated mesenteric and peripheral lymph nodes (FIGS. 4D-4E). In contrast, even after extended colonization, no Th17 cell expansion was observed in K. weizmannii-colonized animals.

To directly gauge the impact of K. weizmannii on host cells the inventors performed an endothelial cell (EC) co-culture assay. EC exposure to C. albicans resulted in EC damage as measured by LDH release. In contrast and similar to co-culture with S. cerevisiae, K. weizmannii did not affect EC viability (FIG. 4F). However, co-infection of EC cultures with K. weizmannii and C. albicans did not prevent EC damage.

Taken together, colonization of Abx-treated animals with both C. albicans and K. weizmannii induced largely cross-reactive humoral immune responses reflected by serum IgA and IgG titers. The characteristic anti-fungal Th17 response was restricted to C. albicans-colonized mice. In combination with the in vitro culture results, these data suggest that K. weizmannii is an innocuous commensal.

### Example 5

### Commensal C. albicans, but not K. weizmannii Causes Pathology in Immune-Suppressed Animals

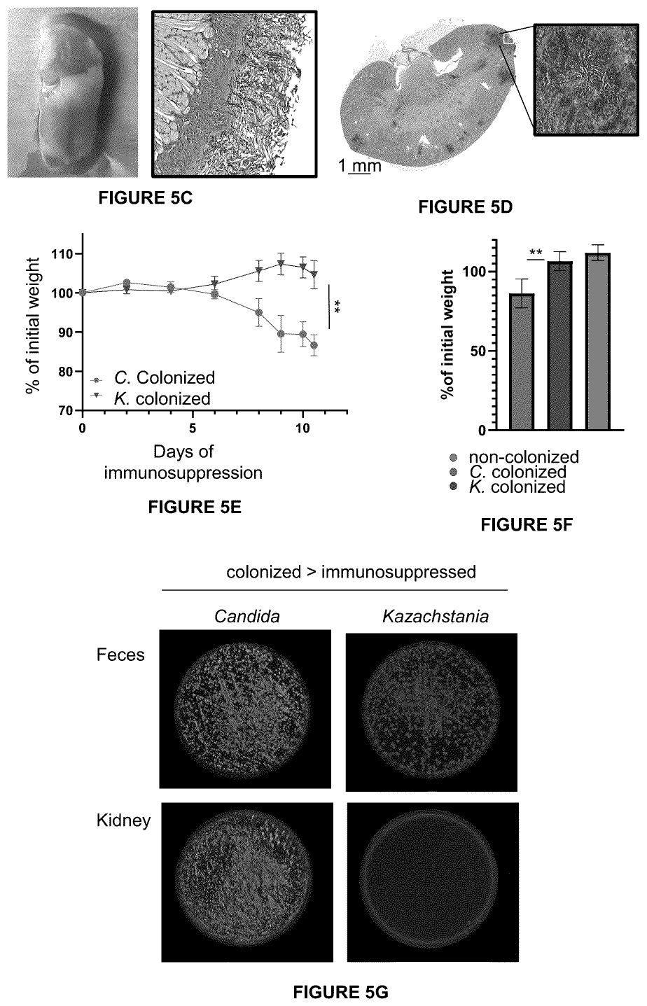

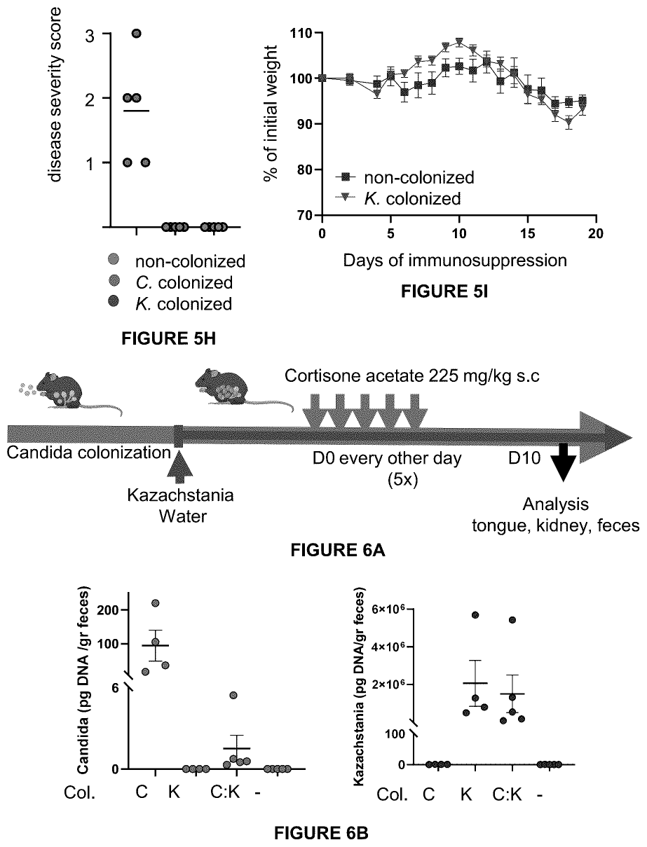

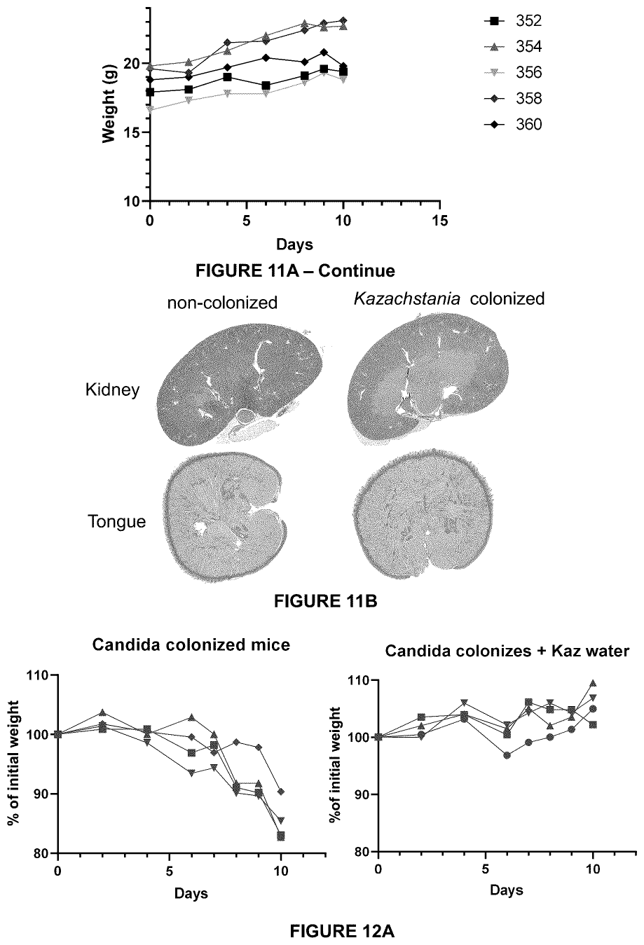

Invasive candidiasis is widely recognized as a major cause of morbidity and mortality in the healthcare environment, often associated with an underlying immune-compromised state. Candidiasis can be induced in otherwise resistant, orally C. albicans-challenged animals by immune-suppression. To test whether corticosteroid treatment would cause C. albicans and K. weizmannii to spread from established commensal reservoirs and cause systemic pathology, the inventors treated mice that were stably colonized with the respective fungi with cortisone 21-acetate boli (225 mg/kg s.c) every other day (FIG. 5A). Unlike control mice, C. albicans-harboring animals lost significant weight after one week of treatment and became moribund (FIG. 5B). Animals displayed prominent tongue candidiasis and fungal growth in the kidneys (FIGS. 5C-5D). In stark contrast, immuno-suppressed animals colonized with K. weizmannii showed neither weight loss nor evidence of fungal spread (FIGS. 5E-5I, and 11A-11B). These data establish that in mice, K. weizmannii is innocuous and even in immunosuppressed animals neither breaches the intestinal barrier to spread systemically nor causes other pathologies.

### Example 6

### Competitive Commensalism Mitigates Candidiasis

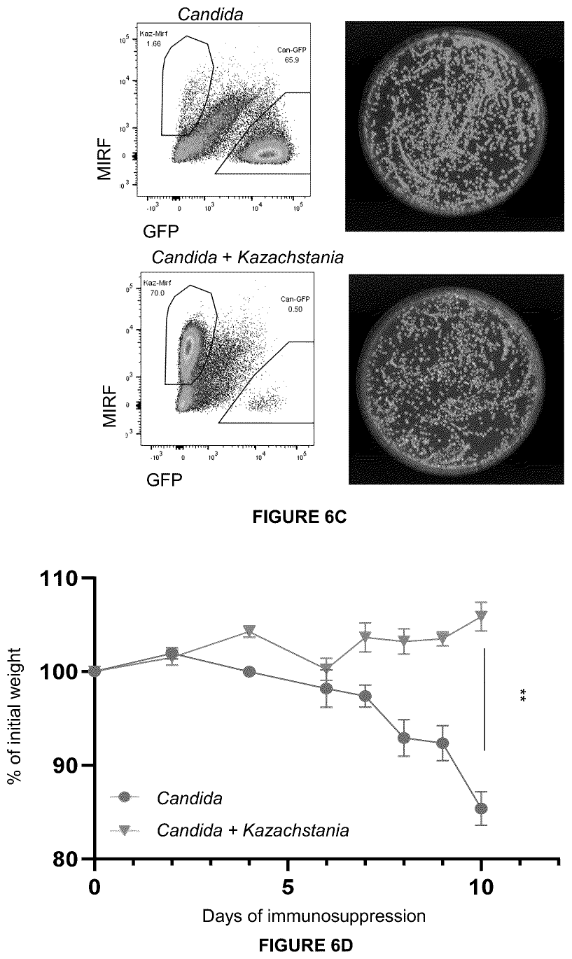

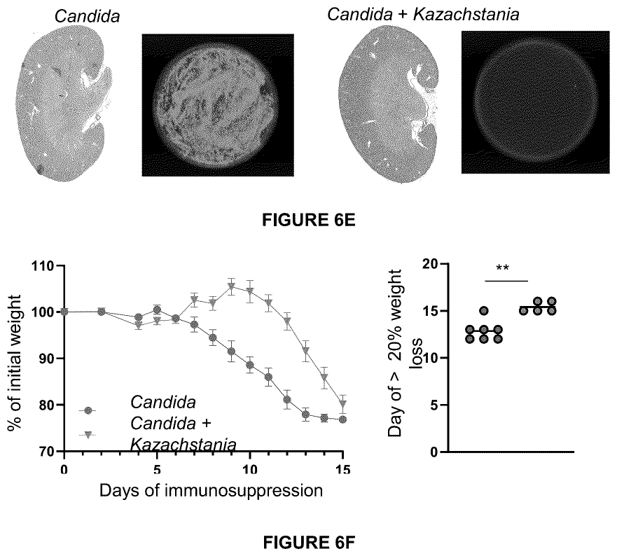

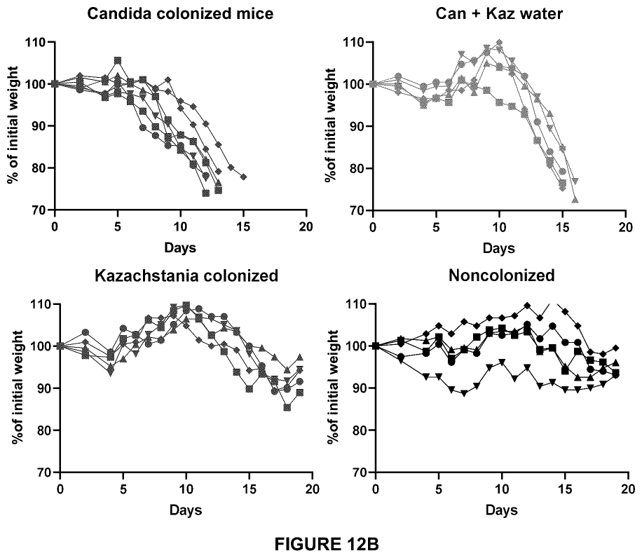

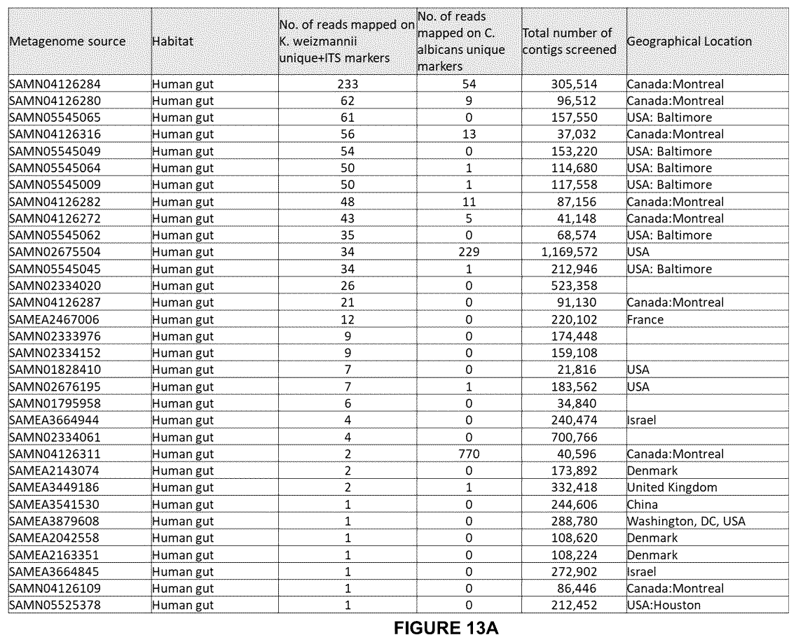

Since K. weizmannii exposure during competitive seeding and cohousing significantly reduces the commensal C. albicans burden in colonized animals (FIG. 3J), the inventors next examined whether this commensal competition would also mitigate candidiasis pathology. To simplify the mode of Kazachstania administration, the inventors treated Candida colonized animals with Kazachstania-supplemented drinking water (FIG. 6A) prior to immunosuppression. As expected, also in this setting Kazachstania efficiently expelled C. albicans from colonized animals (FIG. 6B). Indeed, exposure of immunosuppressed C. albicans-colonized animals to K. weizmannii significantly delayed the weight loss and systemic yeast spread, as indicated by the absence of kidney colonization (FIGS. 6C-6E, and 12A). As to expect, the animals were, however, not permanently protected. Rather, their residual commensal C. albicans burden eventually disseminated and caused pathology (FIGS. 6F-6G, and 12B). Collectively these results establish that K. weizmannii out-competes C. albicans from the commensal microbiome and thereby reducing the cause of pathology, mitigates candidiasis and improves the health status of immunosuppressed animals.

### Example 7

### Kazachstania Presence in Human Metagenomes

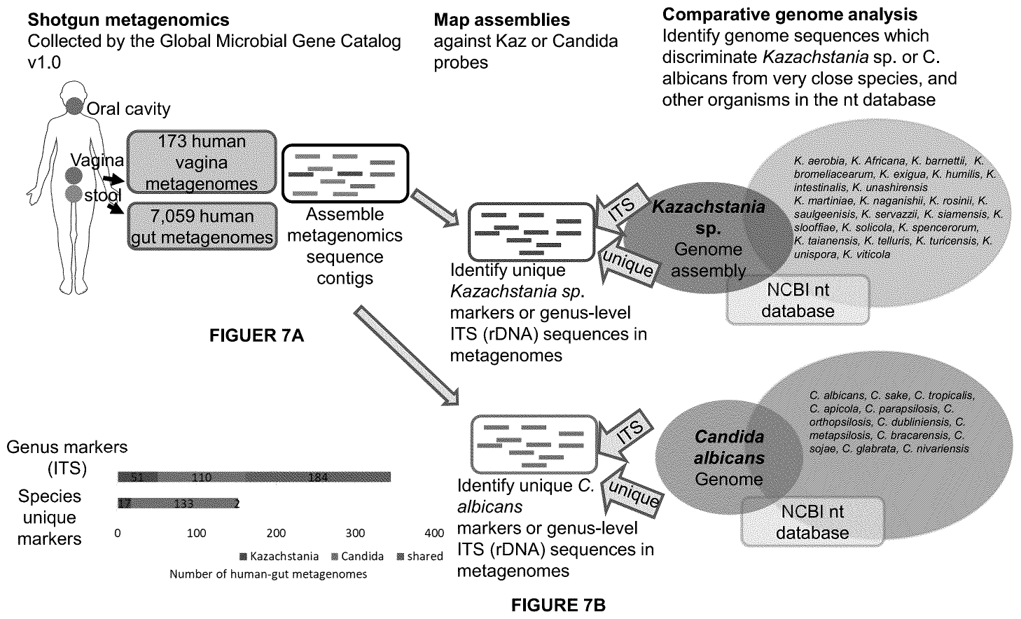

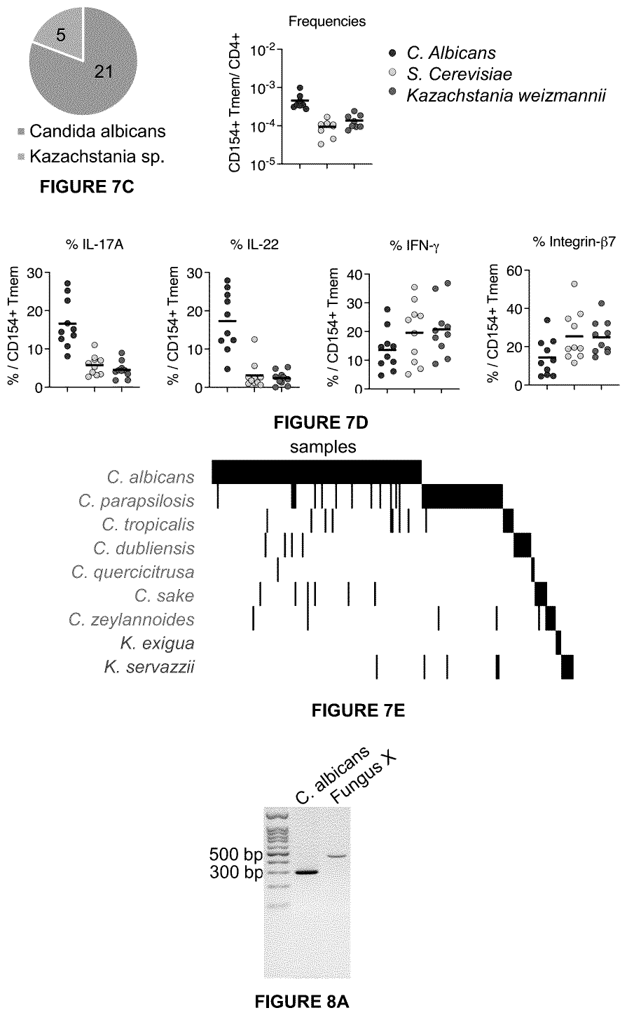

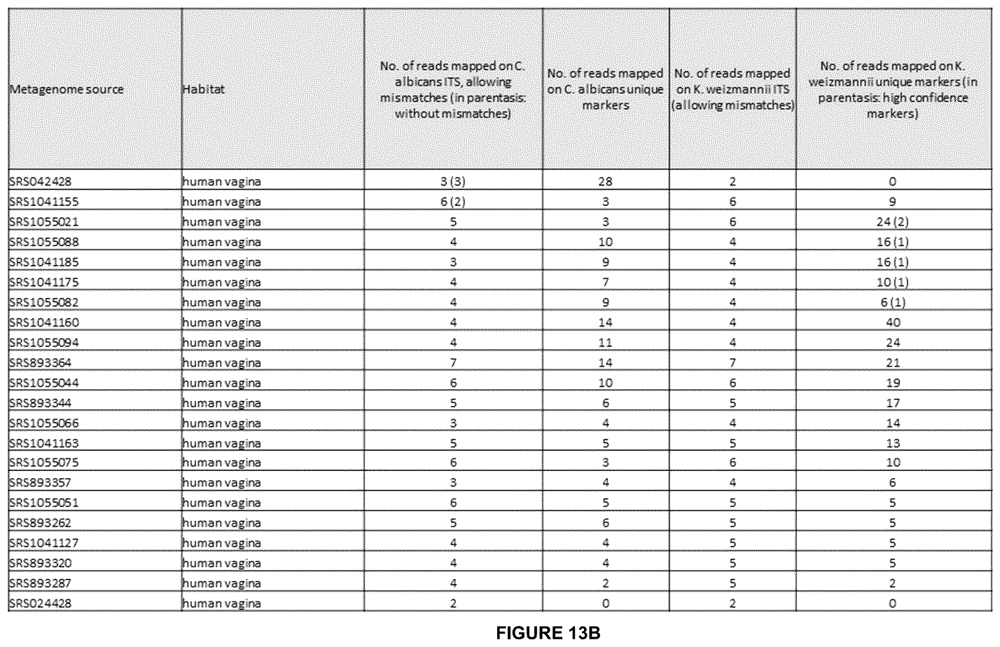

Candida species are a major component of the human mycobiota, with C. albicans being the most prevalent. Despite their established role in dough fermentation, Kazachstania spp. presence in healthy humans or during pathology-associated dysbiosis has very rarely been reported. To gauge the abundance of Kazachstania spp., and in particular, the newly identified K. weizmannii disclosed herein in human microbiota, the inventors designed a bioinformatic screen to detect genomic regions specific to these fungi, out of shotgun sequence information from a collection of 13,174 published metagenomics data sets (FIG. 7A). Among 7,059 human stool samples analyzed, the inventors identified 32 metagenomes that displayed specific evidence for K. weizmannii and 9,236 reads that harbored Kazachstania spp. sequences (26S). 7,167 samples displayed evidence for C. albicans (FIG. 7B). Likewise, among 173 vaginal metagenomes, the inventors found 22 samples with evidence for Kazachstania and Candida sequences, including presence of K. weizmannii (FIGS. 7C, and 13A). These samples had a wide range of biogeographical distribution (FIG. 13B). Collectively, the current data support the notion that Kazachstania spp. are a common and integral part of the human microbiome. The inventors corroborated this notion by an antigen-reactive T cell enrichment (ARTE) analysis of peripheral blood of a limited number of healthy individuals. As shown earlier (Bacher et al., 2019), ARTE revealed T cell reactivity directed against C. albicans extracts, but also against K. weizmannii (FIG. 7D). C. albicans-reactive T cells were polarized towards IL-17- and IL22-producing Th17 fates. In contrast, K. weizmannii-reactive T cells were of the Th1 type, like T cells which reacted to S. cerevisiae. Of note, CD154⁺ memory T cells responsive to Saccharomycetaceae extracts also expressed b7 integrin indicative of their generation in the gut mucosa.

In mice, abundance of C. albicans and K. weizmannii anti-correlated, suggesting that these yeasts compete for similar niches. To investigate the relative distribution of Candida and Kazachstania species in the human microbiome, the inventors performed a sensitive ITS2 analysis of fecal samples of a cohort of 570 healthy individuals (FIG. 7E). Since the Kazachstania genus had not been widely detected in prior ITS2 sequencing initiatives the inventors anticipated that it would be a less prevalent genus with lower abundance when compared with Candida and other prominent genera. Therefore, the inventors added empty control samples which underwent the same amplification and library preparation processes to enable the application of a novel ITS2 processing pipeline previously applied to analyze the low biomass environment of the tumor mycobiome. Indeed, 13 fungal species could be detected in control samples with read numbers ranging from 1-100 reads per species per sample. The inventors, therefore, applied an aggressive cutoff by flooring all species which obtained less than 100 reads in a given sample to zero in that sample. This process yielded 215 samples with ITS2 sequence evidence of either Candida or Kazachstania species.

C. albicans, was the most prominent Candida species found in 120 samples, followed by C. parapsilosis and C. tropicalis, with 29 and 15 samples respectively. Two Kazachstania species were detected across 37 samples prior to flooring, and were completely absent from negative control samples. Still, these species were floored in samples where they did not reach 100 reads leaving 12 samples with Kazachstania servazii and 3 samples with Kazachstania exigua, close relatives of the novel K. weizmannii species. The inventors did not detect K. weizmannii specifically in this limited cohort. Notably, the majority of individuals harboring Kazachstania species displayed mutual exclusive presence with Candida spp. (FIG. 7E) suggesting competitive fungal commensalism, as in mice. Collectively, these data identify Kazachstania spp. as part of the human microbiome and the current data suggest an inverse correlation of these fungi and Candida spp. warranting further studies on a larger scale.

Taken together the herein disclosed K. weizmannii is identified as an innocuous fungal commensal in men and mice. By its virtue to successfully compete with C. albicans in the murine gut for to-be-defined niches, K. weizmannii lowered the pathobiont burden and mitigated candidiasis development in immunosuppressed animals. This competitive fungal commensal is thus suggested as a potential therapeutic for the management of C. albicans-mediated diseases.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. An isolated fungus belonging to the genus Kazachstania and deposited at the ATCC under the deposit number PTA-127318.

2. A composition comprising the isolated fungus of claim 1, and a pharmaceutically acceptable carrier.

3. A composition comprising a fungus, wherein at least 90% of said fungus is the isolated fungus of claim 1.

4.-10. (canceled)

11. A method for preventing or treating an autoimmune disease in a subject in need thereof, the method comprising administering to said subject a therapeutically effective amount of a pharmaceutical composition comprising the isolated fungus of claim 1, thereby preventing or treating an autoimmune disease in the subject.

12. The method of claim 11, wherein said autoimmune disease is: (i) induced T helper 17 cells (Th17) activity; (ii) characterized by increased Th17 activity; or (iii) both (i) and (ii), and optionally wherein said Th17 activity is induced by a fungal infection.

13. (canceled)

14. The method of claim 11, wherein said subject is afflicted with a fungal infection.

15. The method of claim 11, wherein said autoimmune disease is selected from the group consisting of: multiple sclerosis, psoriasis, and asthma.

16. A method for preventing or treating a fungal infection in a subject in need thereof, the method comprising administering to said subject a therapeutically effective amount of a pharmaceutical composition comprising the isolated fungus of claim 1.

17. The method of claim 11, wherein said fungal infection comprises a Candida infection.

18. The method of claim 11, wherein said fungal infection comprises a Candida albicans infection.

19. The method of claim 16, wherein said treating comprises reducing the number or abundance of a fungus inducing said fungal infection in the gastrointestinal tract of said subject.

20. The method of claim 19, wherein said reducing is: (i) by at least 30% compared to control; (ii) for at least 4 days; or (iii) both (i) and (ii).

21. The method of claim 16, wherein said treating comprises reducing the activity, abundance, or both, of Th 17 cells in said subject.

22. The method of claim 11, wherein said administering comprises colonizing the gut of said subject with said isolated fungus.

23. A non-human animal subject comprising the isolated fungus of claim 1.

24. The non-human animal subject of claim 23, wherein said non-human animal subject is gnotobiotic.

25. The non-human animal subject of claim 23, being a mouse.

26. The non-human animal subject of claim 23, wherein the isolated fungus of claim 1 colonizes or is present in the gastrointestinal tract of said non-human animal subject.

27. A method for producing the non-human animal subject of claim 23, comprising colonizing a gastrointestinal tract of a non-human animal subject with an effective amount of an isolated fungus belonging to the genus Kazachstania and deposited at the ATCC under the deposit number PTA-127318, thereby producing the non-human animal subject.

28. The method of claim 27, further comprising rearing/culturing said non-human animal subject under pathogen free conditions.
